(19) United States
(12) Patent Application Publication
(10) Pub. No.: US 2009/0005302 A1
(43) Pub. Date: Jan. 1, 2009

(54) METHOD, APPARATUS, AND COMPOUND FOR EFFECTING LOCALIZED, NON-SYSTEMIC, IMMUNOGENIC TREATMENT OF CANCER

(76) Inventor: Michel Sarkis OBEID, Paris (FR)

Correspondence Address: SAMUEL A. KASSATLY, 20690 View Oaks Way, SAN JOSE, CA, 95120, US

(21) Appl. No.: 11/774,585

(22) Filed: Jul. 7, 2007

(30) Foreign Application Priority Data
Sep. 8, 2006 (EP) 06291427.0-2107

(51) Int. Cl. A61K 38/17; A61K 31/704; A61K 31/675; A61P 35/00

(52) U.S. Cl. 514/12; 514/33; 514/91; 514/27

(57) ABSTRACT

Anthracyclin-treated turn or cells are particularly effective in eliciting an anti-cancer immune response, where the rDNA-damaging agents, such as etoposide and mitomycin C do not induce immunogenic cell death. Anthracyclins induce the rapid, pre-apoptotic translocation of calreticulin (CRT) to the cell surface. Blockade or knock down of CRT suppressed the phagocytosis of anthracyclin-treated tumor cells by dendritic cells and abolished their immunogenicity in mammals, such as mice. The anthracyclin-induced CRT translocation was mimicked by inhibition of the protein phosphatase1/GADD34 complex. Administration of recombinant CRT or inhibitors of protein phosphatase1/GADD34 restored the immunogenicity of cell death elicited by etoposide and mitomycin C, and enhanced their antitumor effects in vivo. These data identify CRT as a key feature determining anti-cancer immune responses and delineate a possible strategy for immunogenic chemotherapy.

### PRIORITY CLAIM

The present application claims the priority of co-pending European patent application, Serial No. 06291427.0-2107, filed on Sep. 8, 2006, titled “Calreticulin For Its Use As A Medication For The Treatment Of A Disease Such As Cancer In A Mammal” which is incorporated herein by reference in its entirety.

### FIELD OF THE INVENTION

The present invention relates to isolated and purified proteins, such as calreticulin, recombinant calreticulin, mimetics of calreticulin or molecules that induce calreticulin exposure at the cell surface for a novel use as a medication in the treatment of diseases such as cancer. Moreover, the invention deals with a new method of treating diseases such as cancers comprising an induction of an immunogenic apoptosis and increased efficiency of chemotherapy. The invention also concerns a method of detection of the calreticulin protein at the cellular surface. A kit for detection the calreticulin at the cellular surface and/or predicting the efficiency of a chemotherapy.

### BACKGROUND OF THE INVENTION

Cancer is a major cause of mortality in most industrialized countries. Different ways of cancer treatment can be used: surgery, radiotherapy, immunotherapy, hormonotherapy and chemotherapy. Numerous research laboratories lead works to find cancer therapy improvements. Chemotherapy leads to the cell death. Two type of cell death are known: the apoptosis and the necrosis.

It has long been hypothesized that apoptotic cell death would be poorly immunogenic (or even tolerogenic) whereas necrotic cell death would be truly immunogenic.

This difference was thought to result from the intrinsic capacity of cells dying from non-apoptotic cell death to stimulate the immune response, for example by stimulating local inflammatory responses (‘danger signals’) and/or by triggering the maturation of dendritic cells (DCs).

In contrast to necrosis (which is defined by brisk plasma membrane rupture), apoptosis is associated with a series of subtle alterations in the plasma membrane that render the dying cells palatable to phagocytic cells. Such “eat me” signals, which include the adsorption of soluble proteins from outside the cell (such as C1q and thrombospondin) and the translocation of molecules from inside the cell to the surface (such as phosphatidylserine, PS, and calreticulin, CRT), as well, as the suppression of “don't eat me” signals (such as CD47) elicit the recognition and removal of apoptotic cells by professional and non-professional phagocytes. Suboptimal clearance of apoptotic cells can trigger unwarranted immune reactions and lead to autoimmune disease.

Nonetheless, it seems that the dichotomy between immunogenic necrosis versus tolerogenic apoptosis is an oversimplification. Thus, unscheduled (necrotic) tumor cell death may induce local immunosuppression. Moreover, the capacity of apoptotic tumor cells to trigger the immune response was found to depend on the apoptosis inducer, leading to the identification of two morphologically undistinguishable subcategories of apoptosis, namely immunogenic versus non-immunogenic.

Most of standard chemotherapies induce a non-immunogenic apoptosis. Thus, even after an initially efficient chemotherapy, patients do not develop an efficient antitumorous immune response and then are overcome by chemotherapy-resistant tumorous variants. To improve anticancer chemotherapy, a promising way is brought by the immunogenic cancer-cell death. Indeed, induction of immunogenic cancer-cell death should be very interesting in that the immune system can contribute through a “bystander effect” to eradicate chemotherapy-resistant cancer cells and cancer stem cells.

The efficiency of a chemotherapy and the responsiveness is relating to drugs used and the molecules involved in the chemotherapy. The main drugs used in anti-tumorous chemotherapy can be divided in four groups: cytotoxic agents, hormones, immune response modulators and inhibitors of the kinase tyrosin activity. Among cytotoxic agents, there are some cytotoxic antibiotics such as anthracyclins (doxorubicin, idarubicin, and mitoxantrone which are apoptosis inducers). It has been shown for the first time that anthracyclins are capable of eliciting immunogenic apoptosis. Indeed, while most apoptosis inducers, including agents that target the endoplasmic reticulum (ER) (thapsigargin, tunicamycin, and brefeldin), mitochondria (arsenite, betulinic acid, C2 ceramide) or DNA (Hoechst 33343, camptothecin, etoposide, mitomycin C), failed to induce immunogenic apoptosis, anthracyclins elicited immunogenic cell death (as shown in FIGS. 1B, C). Despite a growing body of research, under which circumstances an immune response is triggered against dying tumor cells remains an open question.

It has been an ongoing conundrum which particular biochemical change would determine the distinction between immunogenic and non-immunogenic cell death.

The present invention is based on the observation that the protein named calreticulin (CRT) exposure is present on cells that succumb to immunogenic cell death, yet lacks on the surface of cells that undergo non-immunogenic cell death.

CRT has been already described for modulating the hormonal response, another way to treat cancer. Proteins which modulate hormone receptor induced gene transcription are present in the nucleus of the cell and inhibit or promote the binding of a hormone to its receptor. The method described in US publication number US 2003/0060613 A1 presents a purified protein used in modulating hormone responsiveness and efficient in anti-cancer therapy. It describes a synthetic protein, a mimetic protein, a DNA molecule, a method of treating a disease such as cancer and a kit containing a pharmaceutical comprising the protein, mimetic of it or synthetic peptide. The protected protein is the CRT which is present either in the endoplasmic reticulum of a cell or in the nucleus. The publication describes a mechanism of action on gene transcription and thus protects only nuclear CRT.

Actually, one particular alteration was identified in the plasma membrane of dying cells: the surface exposure of calreticulin (CRT). This event only occurs in immunogenic cancer cell death. Exogenous CRT or external provision of signals that induces CRT exposure confers immunogenicity to otherwise non-immunogenic cell death, allowing for an optimal anti-cancer chemotherapy.

Hence, the present invention concerns the calreticulin for its use as a medication for the treatment of a disease in a mammal, said medication inducing an increased location of calreticulin at the cellular surface.

The present invention is based on the identification of CRT exposure as a determining feature of anti-cancer immune responses and delineate a strategy of immunogenic chemotherapy.

The location of the CRT at the cellular surface could be the result of the translocation of intracellular CRT to the cell surface or the result of the translocation of extracellular CRT to the cell surface. So, the present invention concerns the application as a medication wherein the calreticulin (endogenous form or recombinant form or mimetic form) translocation is from the cytoplasm to the membrane of cells or from the extracellular medium to the membrane of cells.

As mimetic form, it should be understood a truncated form of the calreticulin or part(s) of calreticulin or hybrids, exhibiting same properties as native form of calreticulin (i.e., location at the cellular surface).

Furthermore, it has been shown that the calreticulin present in an increased amount at the cell surface renders the dying cells palatable to phagocytic cells such as dendritic cells. These cells interact with the immune system and then induce an immune response, that render the calreticulin as an inducer of immunogenic apoptosis. The present invention also concerns calreticulin for its use as a medication for the treatment of a disease in a mammal said medication inducing an immunogenic apoptosis.

Preferably, by this application as a medication, the disease treated is a cancer such as breast cancer, prostate cancer, melanoma, colon cancer, etc., or an infection like viral or bacterial or fungal or parasitic infection.

The present invention exposes calreticulin for its use as a medication for the treatment of a disease in a mammal, said medication improving the efficiency of chemotherapy in a mammal in need of such chemotherapy by inducing an increased location of CRT at cell surface and/or induction of immunogenic apoptosis.

CRT exposure is known to be induced by UVC light.

But it appears that CRT exposure is also triggered by anthracyclins (as shown in FIG. 2) and PP1/GADD34 inhibitors (as shown in FIG. 5), involving the translocation of intracellular CRT to the cell surface through a molecular mechanism that is not fully understood and that likewise involves the presence of saturable CRT receptors on the cell surface that can bind exogenous CRT as well as endogenous, preformed CRT.

Indeed, it has been shown that this CRT protein was strongly (by a factor of 6) induced by doxorubicin and anthracyclin in general (as shown in FIGS. 2B and 2C). Immunoblot analyses of 2D gels (not shown) and conventional electrophoreses of purified plasma membrane surface proteins (as shown in FIG. 2C) confirmed the surface exposure of CRT after treatment with anthracyclins. This CRT surface exposure was also detectable by immunofluorescence staining of anthracyclin-treated live cells (as shown in FIG. 2D). The induction of CRT exposure by anthracyclins was a rapid process, detectable as soon as 1 h after treatment (as shown in FIG. 1S A, B), and hence preceded the apoptosis-associated phosphatidylserine (PS) exposure (as shown in FIG. 1S C, D). Of note, there was a strong positive linear correlation (p<0.001) between the appearance of CRT at the cell surface (measured at 4 h) and the immunogenicity elicited by the panel of 20 distinct apoptosis inducers (FIG. 2E).

The immunogenicity and the immune response could be mediated by specific cells: dendritic cells (DC). It has been shown that anthracyclin-treated tumor cells acquired the property to be phagocytosed by DC few hours after treatment with doxorubicin or mitoxantrone (as shown in FIG. 3A, supplementary FIG. 2A), correlating with the rapid induction of CRT (as shown in FIG. 3B, FIG. 1S A, B) and the acquisition of immunogenicity (as shown in supplementary FIG. 2B).

Accordingly, blockade of the CRT present on the surface of mitoxantrone-treated cancer cells by means of a specific antibody inhibited their phagocytosis by DC (as shown in FIG. 3C). Inversely, addition of recombinant CRT protein (rCRT), which binds to the surface of the cells, could reverse the defect induced by the blockade of CRT by antibodies. Hence, surface CRT elicits phagocytosis by DC. Moreover, absorption of rCRT to the plasma membrane surface greatly enhanced the immunogenicity of cells that usually fail to induce an immune response such as mitomycin-treated cells (as shown in FIG. 4C) or etoposide-treated cells coated with rCRT and elicited a vigorous anti-tumor immune response in vivo (as shown in FIG. 4D). However, absorption of rCRT to the cell surface without prior treatment with cell death inducers failed to elicit an anti-cancer immune response. The present invention deals with the recombinant calreticulin for its use as a medication for the treatment of a disease in a mammal said medication, after the administration of a cell-death inducer (such as etoposide or mitomycine C), inducing an immunogenic apoptosis.

Accordingly, the present invention also concerns the protein phosphatase inhibitor, as the catalytic subunit of the protein phosphatase 1 (PP1) inhibitor, the GADD34 inhibitor or the complex PP1/GADD34 inhibitor, for its use as a medication for the treatment of a disease in a mammal said inhibitor inducing an increased location of endogenous calreticulin at the cellular surface.

Furthermore, the present invention is directed to the protein phosphatase inhibitor, as the catalytic subunit of the protein phosphatase 1 (PP1) inhibitor, the GADD34 inhibitor or the complex PP1/GADD34 inhibitor, for its use as a medication for the treatment of a disease in a mammal said inhibitor inducing an immunogenic apoptosis by increased calreticulin translocation at the cellular surface (as shown in FIG. 5).

It has been observed that CRT exposure induced by anthracyclins and PP1/GADD34 inhibitors was abolished by latrunculin A, an inhibitor of the actin cytoskeleton and exocytose (as shown in supplementary FIG. 4). They have also 5 shown not only that PP1/GADD34 inhibition induces CRT exposure but also that this process can then improve the anti-tumor immune response.

This present invention also concerns the protein phosphatase inhibitor, as the catalytic subunit of the protein phosphatase 1 (PP1) inhibitor, the GADD34 inhibitor or the complex PP1/GADD34 inhibitor, for its use as a medication for the treatment of a disease in a mammal, said inhibitor improving the efficiency of chemotherapy in a mammal in need of such chemotherapy by inducing an increased location of calreticulin at the cellular surface and/or an immunogenic apoptosis.

Moreover, an amount of such inhibitor of PP1 or GADD34 or the complex PP1/GADD34 described above can be used in a pharmaceutical composition promoting an increased translocation of the calreticulin protein from the cytoplasm to the cell membrane which thus induces an immune response during apoptosis in a mammal.

Said inhibitor-comprised pharmaceutical composition promoting an increased translocation of the calreticulin from the cytoplasm to the cell surface can also ameliorate chemotherapy response in a mammal.

The inhibitor of PP1 or GADD34 or PP1/GADD34 is advantageously chosen among tautomycin, calyculin A or salubrinal.

On the other hand, eIF2α is a typically hyperphosphorylated in endoplasmic reticulum stress due to the activation of stress kinases. It was observed that kinases, known to phosphorylate eIF2α could be involved in the increased calreticulin translocation and exposure at the cell surface. These kinases are the eukaryotic translation initiation factor 2-alpha kinases, for example heme-regulated inhibitor (HRI, also called Hemin-sensitive initiation factor 2-alpha kinase or eukaryotic translation initiation factor 2-alpha kinase 1), protein kinase RNA activated (PKR, also called eukaryotic translation initiation factor 2-alpha kinase 2), PKR-like ER-localized eIF2alpha kinase (PERK, also called eukaryotic translation initiation factor 2-alpha kinase 3) and GCN2 (also called eukaryotic translation initiation factor 2-alpha kinase 4).

The present invention concerns also kinase activator, such as a compound activating one of the eukaryotic translation initiation factor 2-alpha kinases, for example heme-regulated inhibitor (HRI, also called Hemin-sensitive initiation factor 2-alpha kinase or eukaryotic translation initiation factor 2-alpha kinase 1), protein kinase RNA activated (PKR, also called eukaryotic translation initiation factor 2-alpha kinase 2), PKR-like ER-localized eIF2alpha kinase (PERK, also called eukaryotic translation initiation factor 2-alpha kinase 3) and GCN2 (also called eukaryotic translation initiation factor 2-alpha kinase 4), for its use as a medication for the treatment of a disease such as a cancer or a viral infection in a mammal, said activator inducing an increased location of endogenous calreticulin at the cellular surface.

The disease treated by such use of medication comprising this activator is a cancer (breast cancer, prostate cancer, melanoma, colon cancer, etc.) or an infection (viral, bacterial, fungal or parasitic infection).

The present invention also provides the application as a medication, comprising at least calreticulin or inhibitor of PP1, GADD34 or PP1/GADD34 or anthracyclin or an activator of said four kinases or anti-calreticulin antibodies or inhibitory/competitive peptide, said medication improves cancer treatment such as tumors sensitive to VP16/etoposide, radiotherapy or immunotherapy i.e. melanoma, kidney cancer, colon cancer, breast or lung tumors, osteosarcoma.

During autoimmune disorders such as Systemic Lupus Erythematosus (SLE), rheumatoid arthritis, dermatitis, allergy, graft versus host, transplant rejection, or too-strong-immunogenicity in forced apoptosis, etc., it will be interesting to limit the immunogenicity of cell death. It was observed that this could be obtained by a decreased translocation of calreticulin to the cell surface by the use of blocking or neutralizing antibodies anti-calreticulin. An inhibitory or competitive peptide interfering with the translocation of calreticulin could also decrease the amount of calreticulin at the cell surface and then reduce the immunogenicity and the immune response in those diseases.

Thus, the present invention also relates to blocking or neutralizing antibody anti-calreticulin or inhibitory/competitive peptide, interfering with the increased translocation of calreticulin and therefore in the immunogenicity of cell death for its use as a medication for the treatment of autoimmune disorders (SLE, rheumatoid arthritis, dermatitis, etc.), allergy, graft versus host disease, transplant rejection.

In one embodiment of the invention, the anthracyclin as cell death agent can also be used in the preparation of a medication for the treatment of a disease in a mammal said medication inducing an increased location of calreticulin at the cellular surface.

The anthracyclin can also be used in the preparation of a medication for the treatment of a disease such as cancer or viral infection in a mammal, said medication promoting an induction of immunogenic apoptosis by increased calreticulin translocation at the cellular surface.

The present invention also deals with the use of anthracyclin in the preparation of a medication for the treatment of a disease such as cancer or viral infection in a mammal said medication improving the efficiency of chemotherapy in a mammal in need of such chemotherapy by inducing an increased location of calreticulin at the cellular surface and/or an immunogenic apoptosis.

Moreover, the present invention concerns also a pharmaceutical composition which comprises an amount of an anthracyclin promoting an increased translocation of the calreticulin protein from the cytoplasm to the cell membrane which thus induces an immune response during apoptosis in a mammal.

Said anthracyclin-comprised pharmaceutical composition promoting an increased translocation of the calreticulin from the cytoplasm to the cell surface can also improve chemotherapy response in a mammal.

The present invention also provides a method promoting the chemotherapy treatment response in a mammal including administration of the pharmaceutical composition comprising an amount of anthracyclin to a mammal in heed by inducing an increased location of calreticulin at the cellular surface and/or an immunogenic apoptosis. The anthracyclin can be doxorubicin, idarubicin or mitoxantrone.

Although CRT adsorbed to the surface of live cells did enhance their phagocytosis by DC in vitro (as shown in FIG. 3E), CRT had to be combined with a cell death-inducer to elicit a local (as shown in FIG. 4C) or systemic immune response in vivo (as shown in FIG. 4D, FIG. 6). In therapeutics, the combination of a cell death inducer (etoposide or mitomycin C) plus calreticulin (rCRT) was able to cause tumor regression, in immunocompetent (but not in immunodeficient) animals. Similarly, etoposide or mitomycin C could be combined with drugs that induce CRT exposure (salubrinal or tautomycin), leading to stable disease or complete tumor regression in immunocompetent (but not in athymic) hosts (as shown in FIG. 6A B). Live CT26 cells failed to grow in animals that had been cured from CT26 tumors, indicating the establishment of a permanent anti-tumor immune response. As shown here, this knowledge can be employed to stimulate an efficient anti-tumor immune response in which a non-immunogenic chemotherapeutic agent becomes immunogenic when combined with rCRT or PP1/GADD34 inhibitors. These results delineate a strategy of immunogenic chemotherapy for the cure of established cancer such as breast cancer, prostate cancer, melanoma, colon cancer, etc.) or for cure of an infection (viral, bacterial fungal or parasitic infection).

The present invention also concerns a product containing a chemotherapeutic agent and recombinant calreticulin as a combination product for its use in the treatment of disease.

The present invention also deals with product containing a chemotherapeutic agent and the inhibitors (such as the catalytic subunit of the protein phosphatase 1 (PP1) inhibitor, the GADD34 inhibitor or the complex PP1/GADD34 inhibitor) as a combination product for its use in the treatment of disease. This combination product could be used for the treatment of a disease such as a cancer (breast cancer, prostate cancer, melanoma, colon cancer, etc.) or an infection (viral, bacterial fungal or parasitic infection).

The chemotherapeutic agent could be etoposide, mitomycin C, anthracyclin and others well known in therapeutics. The present invention also provides a product of combination described just above (chemotherapeutic agent and calreticulin or cell death agent and said inhibitor) wherein said product improves cancer treatment such as tumors sensitive to VP16/etoposide, radiotherapy or immunotherapy i.e. melanoma, kidney cancer, colon cancer, breast or lung tumors, osteosarcoma.

The present invention would also be directed to a method inducing increased calreticulin translocation from the cytoplasm to the cell surface to enhance an immune response in the apoptosis phenomenon in a mammal, said method comprising administering pharmaceutically effective amount of an inhibitor as the catalytic subunit of the protein phosphatase 1 (PP1) inhibitor, the GADD34 30 inhibitor or the complex PP1/GADD34 inhibitor.

Differently, the present invention includes also a method inducing increased calreticulin translocation from the cytoplasm to the cell surface to enhance an immune response in the apoptosis phenomenon in a mammal, said method comprising administering pharmaceutically effective amount of an anthracyclin.

The increased calreticulin translocation is preferably from cytoplasm to the membrane of tumorous cells.

This method improves cancer treatment preferably those tumors sensitive to VP16/etoposide, radiotherapy, or immunotherapy i.e. melanoma, kidney cancer, colon cancer, breast or lung tumors, osteosarcoma etc. Preferably, this method is directed to treat chemosensitive cancers as much as immunosensitive cancers.

This method shows increased efficiency of chemotherapy in a mammal in need of such chemotherapy.

Preferably, the mammal treated is a human.

Furthermore, the location of calreticulin protein at the cell surface may be realized by antibodies anti-calreticulin which detect the endogenous form of calreticulin, a recombinant form and the mimetic form. A method of detection of all forms of calreticulin protein at the cellular surface is also an object of the present invention. This could be done in vitro, ex vivo or in vivo.

The methods used to detect this calreticulin protein at the cell surface are well-known from the skilled man of the art. These methods comprise immunochemistry on tissue sections (frozen or paraffined), EIA assays such as ELISA on tumor lysates, confocal immunofluoresence or flow cytometry analyses of cytospins, cell aspirates harvested from tumor beds or autoimmune lesions. One object of the present invention is also to develop a method of quantitative detection of the calreticulin (all forms) at the cellular surface. The immunogenicity of the apoptosis is correlated to the amount of calreticulin present at the cell surface. The more calreticulin at the cellular surface, the more immunogenic apoptosis. This method of detection can serve to predict the immunogenicity of the apoptosis. In addition, the effectiveness of a chemotherapy is correlated to the efficiency of the immune response, therefore the immunogenicity of the apoptosis. The more the immunogenic apoptosis, the more the therapeutic efficiency of a chemotherapy. This method of detection of calreticulin at the cell surface can be used for prediction of immunogenic apoptosis and also for therapeutic efficiency of a chemotherapy. The calreticulin in these methods is used as a predictive marker of both immunogenic apoptosis and therapeutic efficiency of a chemotherapy. This method of quantitative detection can also be advantageous to predict risks of forced apoptosis that becomes too immunogenic. Inhibition of the translocation of the calreticulin at the cellular surface could decrease the immunogenicity of the calreticulin and thus reduce or block (in the best case) the immune response.

There is a basal amount of calreticulin at the cellular surface and the increased amount of calreticulin allows to predict an immunogenic apoptosis and/or a therapeutic efficiency of a chemotherapy. By comparison between before and after chemotherapy, the amount of calreticulin at the cell surface is generally largely increased and the increase will be a good predictive marker for immunogenic apoptosis, therapeutic efficiency of a chemotherapy, immunogenic viral infection but also for autoimmune diseases or transplantation rejection/GVH disease (where GVH refers to the transplantation of the bone marrow). The present invention also provides a method of detection of the calreticulin at the cell surface wherein the calreticulin at the cell surface is used as a predictive marker of immunogenic viral infection or autoimmune diseases or transplantation rejection/GVH disease.

Additionally, to detect calreticulin, the present invention also provides a kit of detection of the calreticulin protein at the cell surface, according to the method described above, such kit comprising at least anti-calreticulin antibodies. In an embodiment of the invention, this kit of detection could also bean quantitative one for the detection quantitative of calreticulin at the cellular surface, said kit comprising at least antibodies anti-calreticulin.

The present invention also concerns a kit of prediction of immunogenic apoptosis of tumorous cells and/or of a therapeutic efficiency of a chemotherapy, comprising at least anti-calreticulin antibodies for detection of calreticulin protein at the cell surface. When a large amount of calreticulin is detected, the apoptosis of tumorous cells will be immunogenic and the efficiency of the chemotherapy will be ameliorated. A kit of prediction of immunogenic viral infection or autoimmune diseases or transplantation rejection/GVH disease, said kit comprising at least anti-calreticulin antibodies for detection of calreticulin protein at the cellular surface is also provided by the present invention.

The present invention concerns also a method of detection of the calreticulin protein at the cellular surface for the screening of direct or indirect immunogenic drugs and the method of screening for immunogenic drugs including a step of detection of the calreticulin protein at the cell surface, comprising at least anti-calreticulin antibodies for the screening of direct or indirect immunogenic drugs.

The screening of direct and indirect immunogenic drugs could lead to find more efficient anti-tumorous agents. For the direct method, human tumor cell lines must be chosen and then their incubation with a drug panel should allow to detect if there is a spontaneous CRT translocation from the cytoplasm to the cellular surface by flow cytometry or confocal. For the indirect method of screening, human tumor cell lines were chosen where apoptosis is obtained after etoposide treatment but no CRT translocation at the cellular surface. Then, a drug panel is used to detect which one can reverse this phenomenon. The is used to find new inhibitors of the PP1/GADD34 complex or new activators of kinases GCN2, HRI, PERK, PKR.

### BRIEF DESCRIPTION OF THE DRAWINGS

FIG. 1 shows immunogenic cell death induced by anthracyclins.

FIG. 1A. Frequency of dead and dying cells after treatment with distinct chemotherapeutic agents. CT26 cells were cultured for 24 hours in the presence of the indicated agents for 24-48 h, as described in Materials and Methods, and then were stained with Annexin V-FITC and the vital dye DAPI.

FIG. 1B. Identification of immunogenic cell death inducers. CT26 cells cultured as in FIG. 1A were injected into the left flank, followed by injection of life tumor cells in the right flank 8 days later. The percentage of tumor free mice was determined 120 days later as in FIG. 1C.

FIG. 1C. Incidence of tumors after inoculation of dying cells. The data show the actual frequency of tumor-free mice, for the experiment summarized in FIG. 1B. Day 1 was considered the day of inoculation of dying tumor cells, 1 week before challenge with dying tumor cells.

FIG. 1S: Dissociation of CRT exposure and phosphatidyl serine exposure.

FIG. 1 SA, FIG. 1 SB. Kinetics of CRT exposure. CT26 cells were treated with mitoxantrone for the indicated period, followed by immunofluorescence staining with a CRT-specific antibody and cytofluorometric analysis. Representative pictograms are shown in FIG. 1SA and quantitative data are reported in FIG. 1SB.

FIG. 1SC, FIG. 1SD. Kinetics of PS exposure and cell death. Cells were cultured as in FIG. 1SA and FIG. 1SB for the indicated period, followed by staining with Annexin V (which recognizes phosphatidylserin one the surface of dying cells) plus DAPI (which stains dead cells) and FACS analysis.

FIG. 2: CRT surface exposure in immunogenic cell death.

FIG. 2A-FIG. 2D. Identification of CRT as a surf ace-exposed molecule elicited by anthracyclins. Cells were treated for 4 h with doxorubicin alone (DX) or in combination with Z-VAD-fmk (DXZ), followed by biotinylation of the cell surface and purification of biotinylated proteins, 2D gel electrophoresis (FIG. 2A and inserts in FIG. 2A showing part of the gel at higher magnification) and mass-spectroscopic identification of one doxorubicin-induced spot as CRT (arrows in FIG. 2A and underlined peptides in the CRT protein sequence in FIG. 2B), immunoblot detection of CRT in the plasma membrane protein fraction or the total cell lysate (FIG. 2C) or immunofluorescence detection of CRT on the cell surface (in non-permeabilized live cells) or within the cell (after permeabilization and fixation) (FIG. 2D). Note that the nuclei of untreated cells were visualized with Hoechst 33342 (blue), while those of doxorubicin-treated cells emit a red fluorescence (FIG. 2D). Circles in FIG. 2A indicate the position of ERP57.

FIG. 2E. Correlation between CRT exposure and immunogenicity. The surface exposure of CRT was determined by immunofluororescence cytometry while gating on viable (propidium iodine-negative) cells (inserts) and was correlated with the immunogenicity of cell death (as determined in FIG. 1). CO, control; Tg, thapsigargin; Tu, tunicamycin.

FIG. 2S: FIG. 2S A, FIG. 2S B: Kinetics of phagocytosis and immunogenicity elicited by anthracyclins. CT26 cells were cultured for different periods with mitoxantrone or doxorubicin and then confronted with DC to measure their phagocytosis (FIG. 2SA), as in FIG. 3A or injected into mice, one week before challenge with live cells (FIG. 2SB). Numbers on each column of FIG. 2S B indicate the number of mice that were immunized.

FIG. 3: Requirement of surface CRT for phagocytosis of tumor cells by DC.

FIG. 3A, FIG. 3B. Correlation between tumor cell phagocytosis and CRT exposure. Tumor cells labeled with Cell Tracker Orange were cultured with CD11c-expressing DC and the percentage of DC taking up tumor cells was determined (A) and correlated with the CRT surface exposure (B), measured as in FIG. 2E.

FIG. 3C. Blockade of CRT inhibits DC-mediated phagocytosis. Mitoxantrone treated or control cells were incubated with a blocking chicken anti-CRT antibody, followed by detection of phagocytosis by CD.

FIG. 3D, FIG. 3E, FIG. 3F. Knock-down of CRT inhibits DC-mediated phagocytosis and rCRT restores phagocytosis. Cells were transfected with the indicated siRNAs and optionally treated with rCRT, followed by immunoblot (FIG. 3D) detection of surface CRT (FIG. 3E) and phagocytosis by DC (FIG. 3F). Results are triplicates (X±SD) and representative of three independent experiments. * denotes statistically significant differences using the Student V test at p<0.001.

FIG. 3S: Inhibitory profile of CRT exposure. Cells were treated with mitoxantrone or inhibitors of PP1/GADD34, after pre-incubation for 1 h with the indicated inhibitors of protein synthesis (cycloheximide), RNA synthesis (actinomycin D), microtubuli (nocodazol), or the actin cytoskeleton (latrunculin A). Then, CRT expression was determined by immunocytofluorometry. Results are means of triplicates±SD for one representative experiment out of three.

FIG. 4: CRT is required for the immune response against dying turn or cells.

FIG. 4A. In vivo anti-cancer vaccination depends on CRT. CT26 colon cancer cells 15 were transfected with the indicated siRNAs, then treated with rCRT and/or mitoxantrone (as in FIG. 3D) and the anti-tumor response was measured by simultaneously challenging BALB/c mice with mitoxantrone treated tumor cells in one flank and untreated, live tumor cells in the opposite flank.

FIG. 4B. Priming of T cell responses depending on CRT. CT26 tumor cells were left untransfected or transfected with the indicated siRNAs, then treated with medium alone, mitomycin C or mitoxantrone and injected into the right food pod of Balb/c mice. Five days later, mononuclear cells from the draining popliteal lymph nodes were challenged with freeze-thawed CT26 cells, and IFN-y XXX secretion was assessed at 72 hrs.

FIG. 4C. Exogenous supply of CRT enhances the immunogenicity of CRT-negative dying cells. CT26 cells lacking CRT expression after depletion of CRT with a siRNA and mitoxantrone treatment or after mitomycin treatment were coated with rCRT (inserts) and then injected into the food pad, followed by assessment of the IFN-y secretion by cells from the draining lymph nodes as in FIG. 4B.

FIG. 4D. CRT-mediated amelioration of the immune response against etoposide treated tumor cells. CT26 cells were treated for 24 h with etoposide (or PBS) and rCRT was optionally absorbed to the cell surface (inserts), followed by simultaneous injection of the etoposide±rCRT-treated tumor cells and live tumor cells in opposite flanks and monitoring of tumor growth.

FIG. 5: Induction of calreticulin exposure and immunogenic cell death by inhibition of the PP1/GADD34 complex.

FIG. 5A. CRT exposure after anthracyclin treatment in the absence of a nucleus. Intact cells or enucleated cells (cytoplasts) were treated for 2 hours with mitoxantrone, followed by immunofluorescence detection of CRT exposure. Inserts show the effective removal of Hoechst 33342-stainable nuclei from the cytoplasts.

FIG. 5B. Phosphorylation of eIF2α after treatment with anthracyclins. Cells were treated for four hours with mitoxantrone or doxorubicine followed by immunoblot detection of phosphorylated eIF2α irrespective of its phosphorylation state and GAPDH as a loading control.

FIG. 5C-FIG. 5D. Induction of CRT exposure by knock-down of PP1. Cells were transfected with siRNAs specific for the indicated transcripts and were treated 36 h later for 2 h with mitoxantrone prior to immunoblot (FIG. 5C) and cell surface staining (FIG. 5D).

FIG. 5E—Kinetics of CRT exposure determine by FACS analyse after incubation of cells with the indicated agents.

FIG. 5F-FIG. 5G PP1/GADD34 inhibitors render cell immunogenic via CRT. Tumor cells were first transfected with a control siRNA or a CRT-specific siRNA and then treated in vitro with etoposide, alone or in combination with PP1/GADD34 inhibitors. Two hours later, the surface CRT was detected to demonstrate the effective expression of CRT on control siRNA-transfected cells treated with etoposide alone or etoposide plus PP1/GADD34 inhibitors (FIG. 5F), and later, the cells were injected as in FIG. 1A to determine their capacity to inhibit the growth of live tumor cells inoculated one week later (FIG. 5G). The results represent the % of tumor free mice (comprising a total of 12 to 18 mice per group).

FIG. 6: FIG. 6A, FIG. 6B, FIG. 6C. Therapeutic effect of CRT or PP1/GADD34 inhibitors injected into tumors. CT26 tumors established in immunocompetent wild type (FIG. 6A) or athymic nu/nu Balb/c mice (FIG. 6B) were injected locally with the indicated combinations of mitoxantrone, etoposide, mitomycin C, rCRT, salubrinal or tautomycin, followed by monitoring of tumor growth. Each curve represents one mouse. Numbers in the lower right corner of each graph indicate the number of mice that manifest complete tumor involution at day 45. FIG. 6C. Identical experimental setting using intratumoral etoposide plus contralateral subcutaneous injection of rec.CRT. The graphs depict one representative experiment out of two, comprising 5 mice/group.

### DETAILED DESCRIPTION OF PREFERRED EMBODIMENTS

Materials and Methods

Cell Lines and Cell Death Induction.

CT26 cells were cultured at 37° C. under 5% CO2 in RPMI 1640 medium supplemented with 10% FCS, penicillin, streptomycin, 1 mM pyruvate and 10 mM HEPES in the presence of doxorubicin (DX; 24 h, 25 μM), mitoxantrone (Mitox; 24 h, 1 μM, Sigma), idarubicin (24 h, 1 μM, Aventis, France), mitomycin C (30 μM, 48 h; Sanofi-Synthelabo, France), and/or zVAD-fmk (50 μM, 24 h; Bach em), tunicamycin (24 h, 65 μM), thapsigargin (24 h, 30 μM), brefeldin A (24 h, 50 μM, Sigma), etoposide (48 h, 25 μM, Tava classics), MG132 (48 h, 10 μM), ALLN (48 h, μM), betulinic acid (24 h, 10 μM), Hoechst 33343 (24 h, 0.2 μM), camptothecine (24 h, 15 μM), lactacystin (48 h, 60 μM), BAY 11-8072 (24 h, 30 μM), staurosporine (24 h, 1.5 μM), bafilomycin A1 (48 h, 300 μM), arsenic trioxide (24 h, 30 nM), C2-ceramide (C2-C; 24 h, 60 μM), calyculin A (48 h, 30 nM), or tautomycin (48 h, nM, Sigma) and/or salubrinal (48 h, μM).

Cell Death Assays.

Cells were trypsinized and subjected to cytofluorometric analysis with a FACS Vantage after staining with 4,6-diamino-2-phenylindole (DAPI, 2.5 mM, 10 min, Molecular Probes) for determination of cell viability, and Annexin V conjugated with fluorescein isothiocyanate) for the assessment of phosphatidylserine exposure.

siRNAs and Manipulation of Surface CRT.

siRNA heteroduplexes specific for CRT (sense strand: 5-rCrCrGrCrUrGrGrGrUrCrGrArArUrCrRrArATT-3′), GADD34 5′-(rCrArGrGrArGrCrArGrArUrCrArGrArUrArGrATT-3), PPI Cα (5′-rGrCrU rGrGrCrCrUrArUrArArGrArUrCrArGrATT-3′) or on unrelated control (5′rGrCrCrGrGrUrArUrGrCrCrGrGrUrUrArArGrUTT-3′) were designed and synthesized by Sigma-Proligo. CT26 cells were transfected by siRNAs at a final concentration of 100 nM using HiPerFect. Thirty six hours post-transfection CT26 cells were assessed for total CRT content by immunoblotting. To restore CRT expression, cells were exposed to rCRT, produced as described, at 3 μg/10⁶ cells in PBC on ice for 30 min, followed by three washes.

Fluorescence Detection of Cell Surface CRT.

CT26 cells (on a glass slide or in 12-well plates) were first washed with FACS buffer (1×PBS, 5% fetus bovine serum, and 0.1% sodium azide) and then incubated with rabbit anti-mouse CRT antibody (1:100, Stressgen) in FACS buffer at 4° C. for 30 min. Cells reacted with anti-rabbit IgG (H+L) Alexafluor 488-conjugates (1:500) in FACS buffer at 4° C. for 30 min. After washing three times with FACS buffer, surface CRT was detected by cytofluorometric analyse on a FACS Vantage. In some experiments, cells were fixed with 4% paraformaldehyde, counterstained with Hoechst (2 μM; Sigma), followed by fluorescence microscopic assessment.

Immunoblot Analyses.

Cells were washed with cold PBS at 4° C. and lysed in a buffer containing 50 mM Tris HCl pH 6.8, 10% glycerol and 2% SDS. Primary antibodies detecting CRT (dilution 1/2000), CD47 (dilution 1/500), eIF2α, eIF2α-P, and PP1cα (dilution 1/2000), and GADD34 (dilution 1/2000), were revealed with the appropriate horseradish peroxidase-labeled secondary antibody and detected by ECL. Anti-actin or anti-GAPDH was used to control equal loading.

Anti-tumor Vaccination and Treatment of Established Tumors.

All animals were maintained in specific pathogen-free conditions and all experiments followed the FELASA guidelines. 3×10⁶ treated CT26 cells were inoculated s.c. in 200 ml of PBS into BALB/c six-week-old female mice, into the lower flank, while 5×10⁵ untreated control cells were inoculated into the contralateral flank. For the tumorigenicity assay, 3×10⁶ treated or untreated CT26 cells were injected s.c. into nu/nu mice. To assess the specificity of the immune response against CT26, injections of either 5×10⁵ or 5×10⁶ of CT26 were made (for the mice immunized in a standard protocol or vaccination protocol, respectively). Tumors were evaluated weekly, using a caliper. In a series of experiments, BALB/c (wild type or nu/nu) carrying palpable CT26 tumors (implanted 14 days before for wild type or 7 days before for nu/nu mice by injection of 10⁶ tumor cells) received a single intratumoral injection of 100 μM PBS containing the same concentration of anti-cancer agents and PP1/GADD34 inhibitors as those used in vitro, as well as rCRT (15 μg). For the assessment of local immune response, 3×10⁶ cells were injected in 50 μl into the footpad of mice. Five days later, mice were sacrificed and the draining lymph nodes were harvested. 1×10⁵ lymph node cells were cultured for 4 days alone or with 1×10⁶ CT26 cells killed by a freeze-thaw cycle in 200 μl in round-bottom 96-well plates. IFN-γ was determined by ELISA.

Generation of BMDCs.

BM cells were flushed from the tibias and femurs of BALB/c mice with culture medium composed of RPMA 1640 medium supplemented with 10% heat-inactivated FBS, sodium pyruvate, 50 mM 2-ME, 10 mM HEPES (pH 7.4), and penicillin/streptomycin. After one centrifugation, BM cells were resuspended in Tris-ammonium chloride for 2 min to lyse RBC. After one more centrifugation, BM cells (1×10⁶ cells/ml) were cultured in medium supplemented with 100 ng/ml recombinant mouse FLT3 ligand in 6-well plates. After 7 days, the non-adherent and loosely adherent cells were harvested with Versene, washed and transferred in 12-well plates (1.5×10⁶ cells/plate) for cocultures with tumor cells.

Phagocytosis Assays.

In 12-well plates, 25×10⁶ adherent CT26 cells were labeled with Celltracker Orange and then incubated with drugs. In some experiments viable CT26 were coated with 2 μg/10⁶ cells of chicken anti-CRT antibody (ABR affinity bioreagents) or an isotype control for 30 minutes prior to washing and feeding to dendritic cells Cs. Alternatively CT26 cells were coated with 3 μg/10⁶ cells of rCRT on ice for 30 minutes and washed twice prior to addition to dendritic cells. Cells were then harvested, washed three times with medium supplemented with FBS and cocultured with immature DC for 2 hours at a ratio of 1:1 and 1:5. At the end of the incubation, cells were harvested with Versene, pooled with non-adherent cells present in the supernatant, washed and stained with CD11c-FITC antibody. Phagocytosis was assessed by FACS analysis of double positive cells. Phagocytic indexes refer to the ratio between values obtained at 4° C. and values obtained at 37° C. of co-incubation between DC and tumor cells.

Statistical Analyses.

Data are presented as arithmetic mean±standard deviation (SD) or percentages. The t-test was used to compare continuous variables (comparison of tumor growth), the Chi square test for non-parametrical variables (comparison of animal cohorts). For all tests, the statistical significance level was set at 0.05.

Biochemical Methods.

The purification of plasma membrane proteins, mass spectroscopy and the generation of cytoplasts are detailed below.

Biotinylation of GT26 Cell Surface Proteins.

Biotinylation and recovery of cell surface proteins were performed with a method adapted from Gottardi et al. (Gottardi, C. J. et al., “Biotinylation and assessment of membrane polarity: caveats and methodological concerns,” Am J Physiol 268, F285-295 (1995)) and Hanwell et al (Hanwell, D. et al., “Trafficking and cell surface stability of the epithelial Na+ channel expressed in epithelial Madin-Darby canine kidney cells,” J Biol Chem 277, 9772-9779 (2002)). Briefly, 20×10⁶ CT26 cells grown on 75 cm² flask were placed on ice and washed three times with ice-cold PBS-Ca²⁺—Mg²⁺ (PBS with 0.1 mM CaCl2 and 1 mM MgCl2). Membrane proteins were then biotinylated by a 30-minute incubation at 4° C. with NHS-SS-biotin 1.25 mg/ml freshly diluted into biotinylation buffer (10 mM triethanolamine, 2 mM CaCl2, 150 mM NaCl, pH 7.5) with gentle agitation. CT26 cells were rinsed with PBS-Ca²⁺—Mg²⁺+ glycine (100 mM) and washed in this buffer for 20 minutes at 4° C. to quench unreacted biotin. The cells were then rinsed twice with PBS-Ca²⁺—Mg²⁺, scraped in cold PBS, and pelleted at 2,000 rpm at 4° C. The pellets were solubilized for 45 min in 500 μl of lysis buffer (1% Triton X-100, 150 mM NaCl, 5 mM EDTA, 50 mM Tris, pH 7.5) containing protease inhibitors. The lysates were clarified by centrifugation at 14,000×g for 10 min at 4° C., and the supernatants were incubated overnight with packed streptavidin-agarose beads to recover biotinylated proteins. The beads were then pelleted by centrifugation, and aliquots of supernatants were taken to represent the unbound, intracellular pool of proteins. Biotinylated proteins were eluted from the beads by heating to 100° C. for 5 minutes in SDS-PAGE sample buffer before loading onto a 10% SDS-PAGE gel as described above. To ensure the absence of leakage of biotin into the cells, the absence of the intracellular protein actin and GAPDH in biotinylated extracts was systematically verified.

2D Gel Electrophoresis Analysis and Protein Identification by Mass Spectrometry.

Purified proteins were precipitated using the Ettan 2-D clean up kit were subsequently resuspended in urea buffer (7M urea, 2M thiourea, 2% Chaps, 1% Sulfobetaine SB3-10, 1% Amidosulfobetaine ASB14, 50 mM DTT). For the first dimension of protein separation, isoelectric focusing (IEF) was performed using 18-cm immobilized nonlinear pH gradient strips (pH 3 to 10; GE Healthcare) on a electrophoresis unit. Proteins (100 μg) were loaded by in-gel rehydratation for 9 h, using low voltage (30V) then run using a program in which the voltage was set for 1 h at 100 V, 2 h at 200 V, 1 h at 500 V, 1 h at 1,000 V, 2 hrs, 2 hrs voltage gradient 1,000-5,000V and 4 h at 8,000 V. Prior to the second-dimension electrophoresis, IPG gel strips were equilibrated for 10 min at room temperature in 1% dithiothreitol to reduce the proteins and sulfhydryl groups were subsequently derivatized using 4% iodoacetamide (both solutions were prepared in 50 mM Tris (pH 8.8)-6 M urea-30% glycerol-2% SDS-2% bromophenol blue). Strips were transferred to 1.0-mm-thick 10% (wt/vol) polyacrylamide gels (20 by 20 cm), and the second-dimension gels were run at 50 μA for 6 hours. Gels were stained with Sypro Ruby and visualized using a scanner. The analyser was used for matching and analyse of visualized protein spots among differential gels. Background subtraction was used to normalize the intensity value representing the amount of protein perspot.

Differentially expressed spots were excised from the gels with an automatic spot picker placed in Eppendorf tubes, and destained by washing for 5 min with 50 μL of 0.1 M NH4HCO3. Then 50 μL of 100% acetonitrile were added incubated for other 5 minutes. The liquid was discarded, the washing steps were repeated one more time and gel plugs were shrunk by addition of pure acetonitrile. The dried gel pieces were reswollen with 4.0 ng/μL trypsin in 50 mM NH4HCO3 and digested overnight at 37° C. Peptides were concentrated with ZipTip® μC18 pipette tips. Co-elution was performed directly onto a MALDI target with 1 μL of α-cyano-4-hydroxycinnamic acid matrix (5 μg/mL in 50% acetonitrile, 0.1% TFA). MALDI-MS and MALDI-MS/MS were performed on an analyzer with TOF/TOF ion optics. Spectra were acquired in positive MS reflector mode and calibrated either externally using five peaks of standard or internally using porcine trypsin autolysis peptide peaks (842.51, 1045.56 and 2211.10 (M+H)⁺ ions). Mass spectra were obtained from each sample spot by 30 sub-spectra accumulation (each including 50 laser shots) in a 750 to 4000 mass range. Five signal-to-noise best peaks of each spectrum were selected for MS/MS analysis. For MS/MS spectra, the collision energy was 1 keV and the collision gas was air.

MS and MS/MS data were interpreted using a software that acts as an interface between the database containing raw spectra and a local copy of a search engine. Peptide mass fingerprints obtained from MS analysis were used for protein identification in a non-redundant database. All peptide mass values are considered monoisotopic and mass tolerance was set <50 ppm. Trypsin was given as the digestion enzyme, 1 missed cleavage site was allowed, methionine was assumed to be partially oxidized and serine, threonine and tyrosine partially phosphorylated. Scores greater than 71 were considered to be significant (p<0.005). For MS/MS analysis, all peaks with a signal-to-noise ratio greater than 5 were searched against the database using the same modifications as the MS database. Fragment tolerance less than 0.3 Da was considered.

### Preparation of Cytoplasts.

Trypsinized CT26 cells were enucleated as described. Briefly, cells were treated in 2 ml of complete RPMI medium containing cytochalasin B (10 μg/ml; Sigma) and DNase I (80 U/ml; Sigma). Cell suspension was adjusted to a final concentration of 5×10⁶/ml and incubated at 37° C. for 45 minutes before being layered onto a previously prepared discontinuous Ficoll density gradient (3 ml of 100%, in 1 ml of 90% and 3 ml of 55% Ficoll Paque layer containing 5 μg/ml cytochalasin B and 40 U/ml DNase I; gradients were prepared in ultracentrifuge tubes and pre-equilibrated at 37° C. in a CO2 incubator overnight). Gradients containing cell suspensions were centrifugated in a prewarmed SW41 Beckman rotor at 25 000 rpm for 20 minutes at 30° C. The cytoplasts-enriched fraction was collected from the interface between 90 and 100% Ficoll layers, washed in complete RPMI 1640 medium, and incubated at 37° C. The cells were incubated with mitoxantrone (MTX), calyculin (CA), salubrinal (Sal) and tautomycin (TA) for the period of time indicated in the experiment. Then the cell surface CRT was detected (see materials and methods) and the viability was determined by with propidium iodine staining (2 μg/ml, Sigma) for 5 min followed by cytofluorometric analyse. Alternatively cythoplasts were cocultured with immature DC for 2 hours at a ratio of 1:1 and 1:5. At the end of the incubation, cells were harvested with versene, pooled with non-adherent cells present in the supernatant, washed and stained with CD11c-FITC antibody. Phagocytosis was assessed by FACS analyse of double positive cells.

The following examples provide some illustrations of the present invention.

### Example 1

### CRT Exposure Defines Immunogenic Cell Death

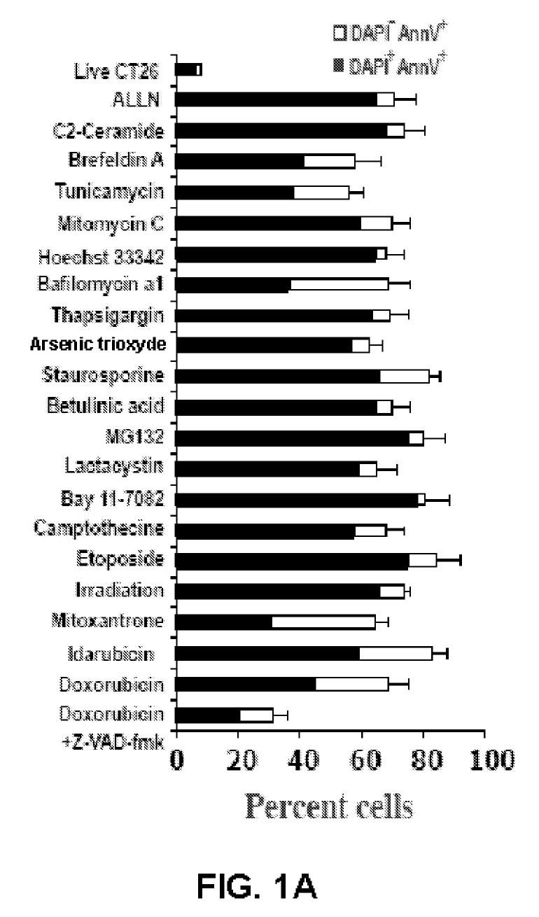

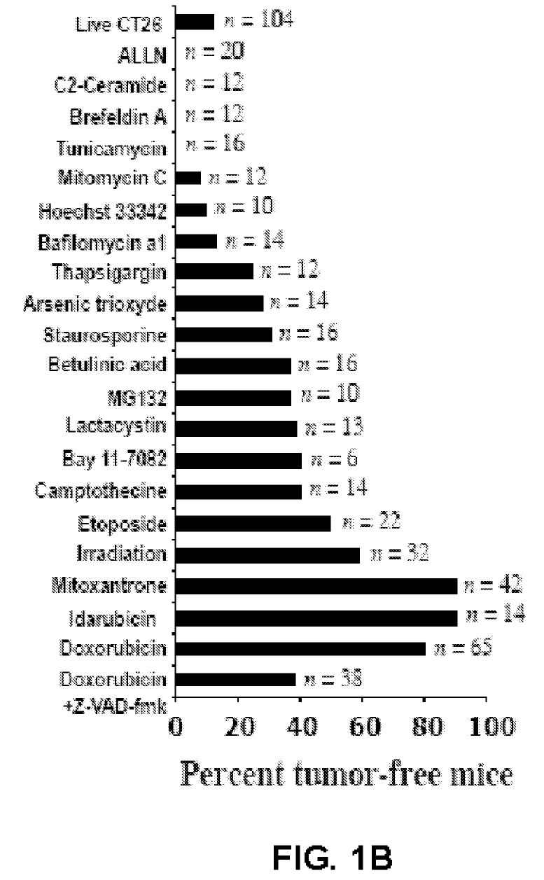

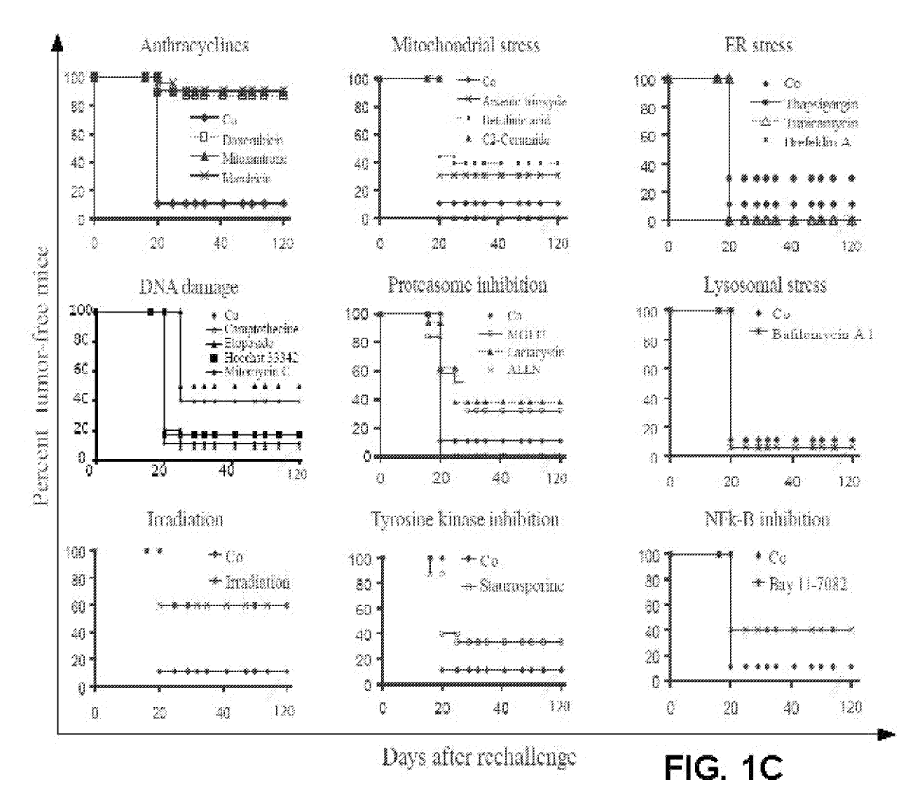

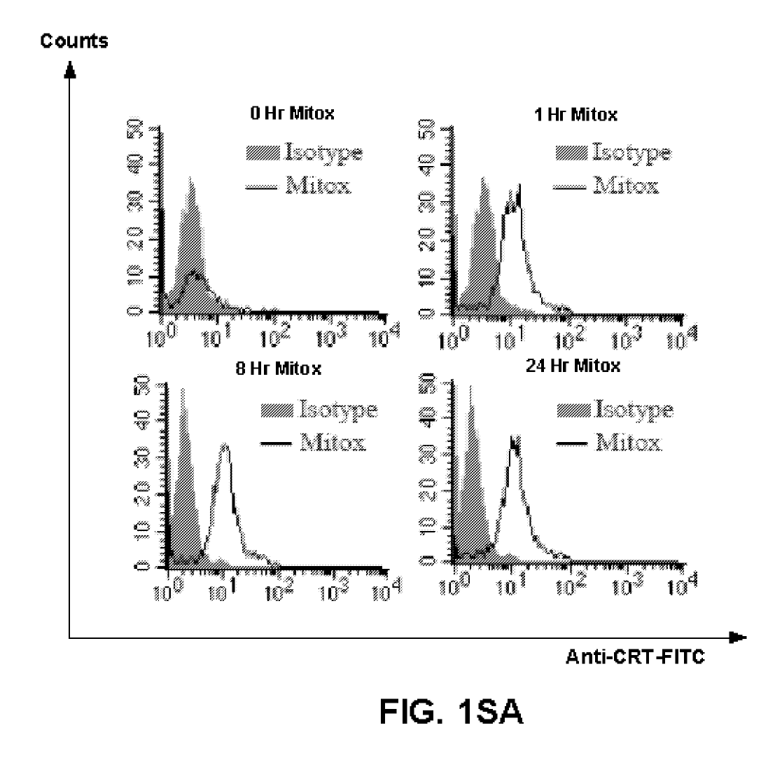

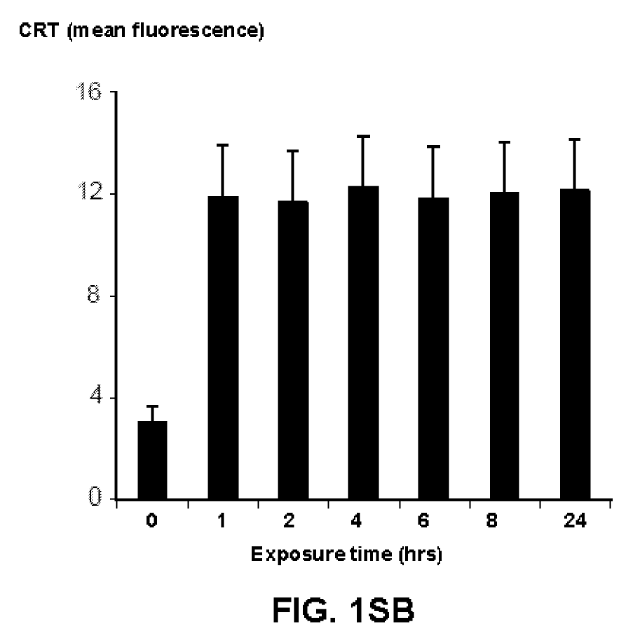

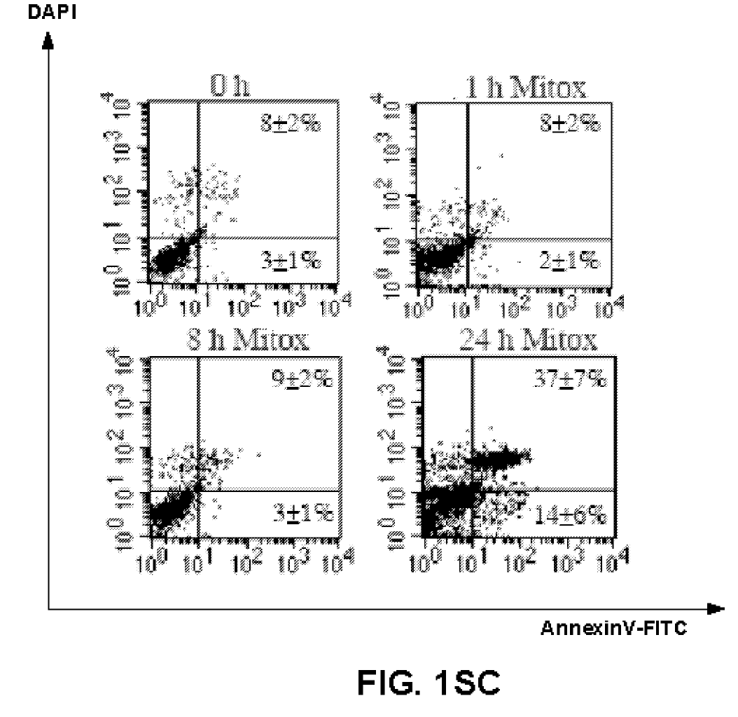

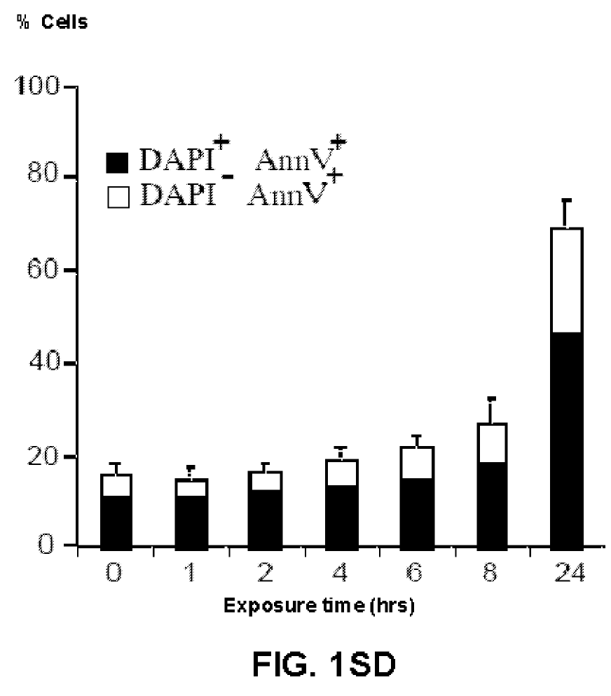

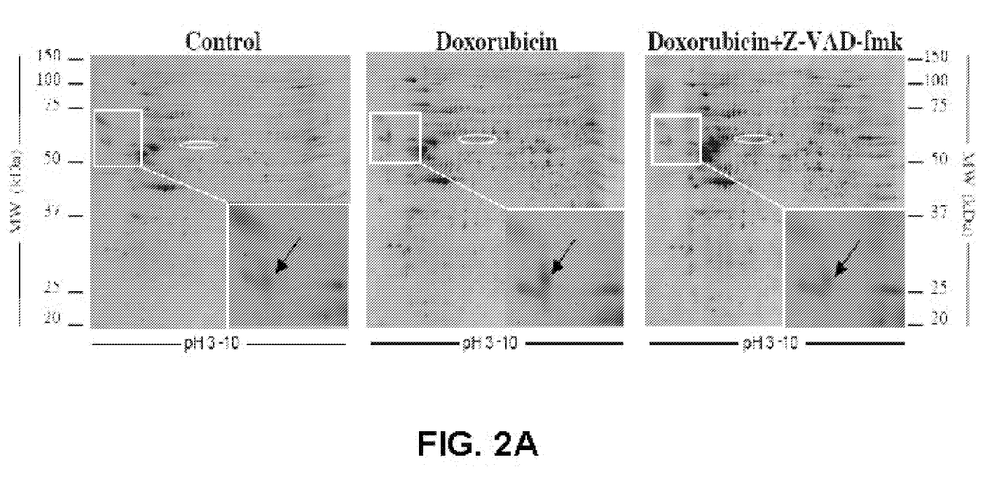

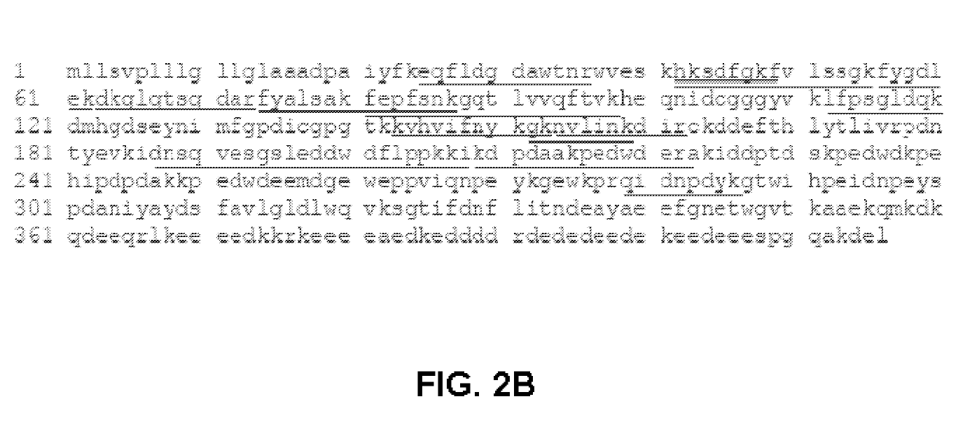

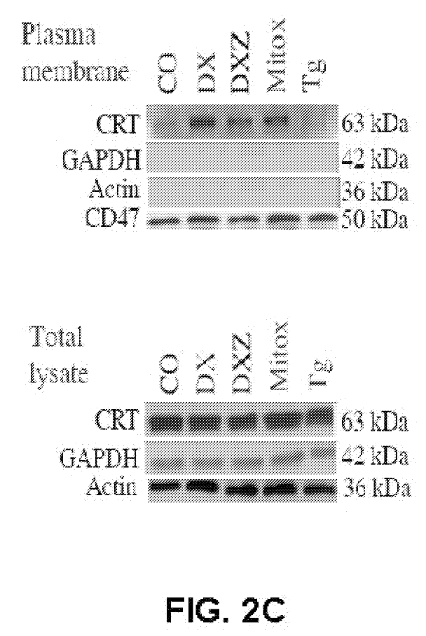

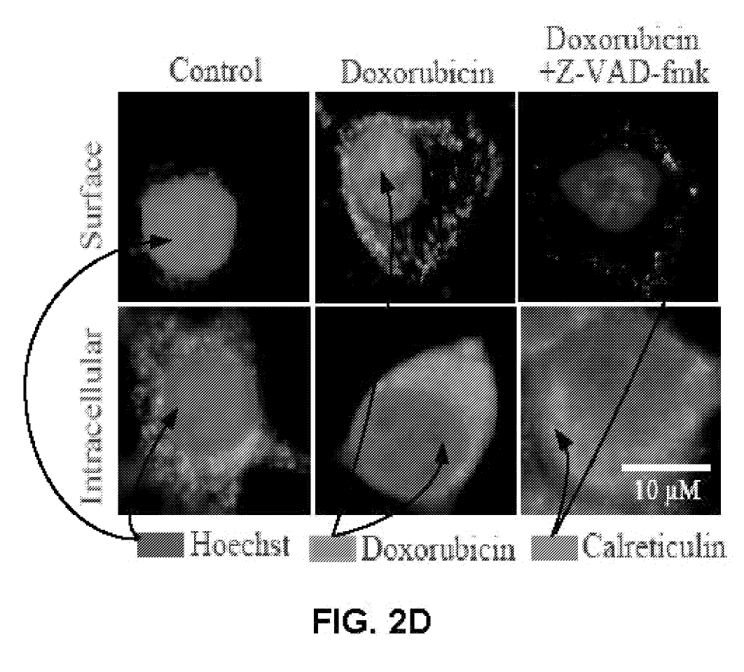

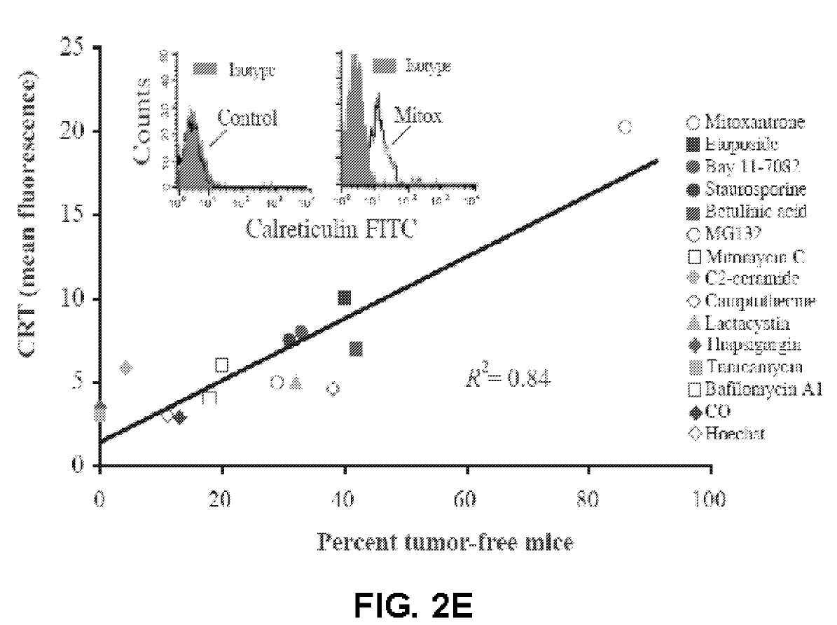

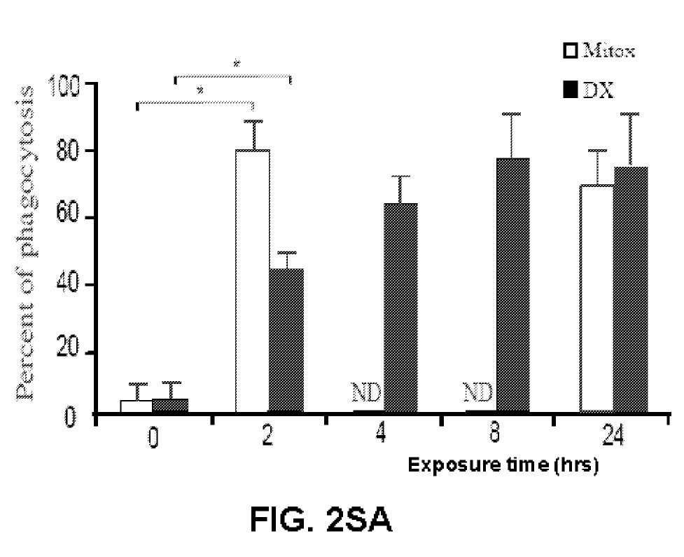

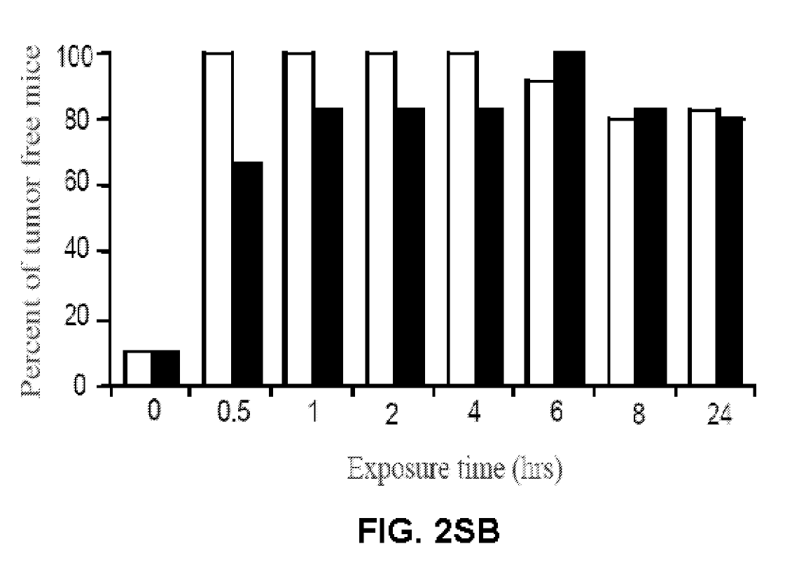

Dying CT26 tumor cells exposed to a panel of −20 distinct apoptosis inducers (all of which induced −70±10% apoptosis, as determined by double staining with the vital dye DAPI and the PS-binding dye Annexin V, FIG. 1A) were injected into one flank of immunocompetent BALB/c mice, followed by rechallenge of the animals with live tumor cells injected into the opposite flank 8 days later. Protection against tumor growth then was interpreted as a sign of anti-tumor vaccination (FIG. 1B) because such protection was not observed in athymic (nu/nu) BALB/c mice. Most apoptosis inducers, including agents that target the endoplasmic reticulum (ER) (thapsigargin, tunicamycin, brefeldin), mitochondria (arsenite, betulinic acid, C2 ceramide) or DNA (Hoechst 33342, camptothecin, etoposide, mitomycin C), failed to induce immunogenic apoptosis, while anthracyclins (doxorubicin, idarubicin and mitoxantrone) elicited immunogenic cell death (FIGS. 1B, C). To identify changes in the plasma membrane proteome, biotinylated surface proteins were affinity-purified from cells that were either untreated or short-term (4 h) treated with doxorubicin or doxorubicin plus Z-VAD-fmk, a pan-caspase inhibitor that reduces the immunogenicity of doxorubicin-elicited cell death (FIG. 1B). Comparison of 2D electrophoreses (FIG. 2A), followed by mass spectroscopic analyses, led to the identification of CRT (FIG. 2B) as a protein that was strongly (by a factor of 6) induced by doxorubicin, but less so (by a factor of 1.3) by doxorubicin combined with Z-VAD-fmk. Another protein whose surface exposure was specifically induced by doxorubicin were identified as ERP57 (FIG. 2A), a CRT-interacting chaperone. Immunoblot analyses of 2D gels and conventional electrophoreses of purified plasma membrane surface proteins (FIG. 2C) confirmed the surface exposure of CRT after treatment with anthracyclins. This CRT surface exposure was also detectable by immunofluorescence staining of anthracyclin-treated live cells (FIG. 2D) and was not accompanied by a general increase in the abundance of intracellular CRT (FIGS. 2C, 2D). The induction of CRT exposure by anthracyclins was a rapid process, detectable as soon as 1 hour after treatment (FIGS. 1S A-B), and hence preceded the apoptosis-associated phosphatidylserine (PS) exposure (FIG. 1S CD). CRT exposure did not correlate with alterations in CD47 expression (FIG. 2C). Of note, there was a strong positive linear correlation (p<0.001) between the appearance of CRT at the cell surface (measured at 4 hours) and the immunogenicity elicited by the panel of 20 distinct apoptosis inducers (FIG. 2E).

### Example 2

### Requirement of CRT for DC-Mediated Recognition of Dying Tumor Cells

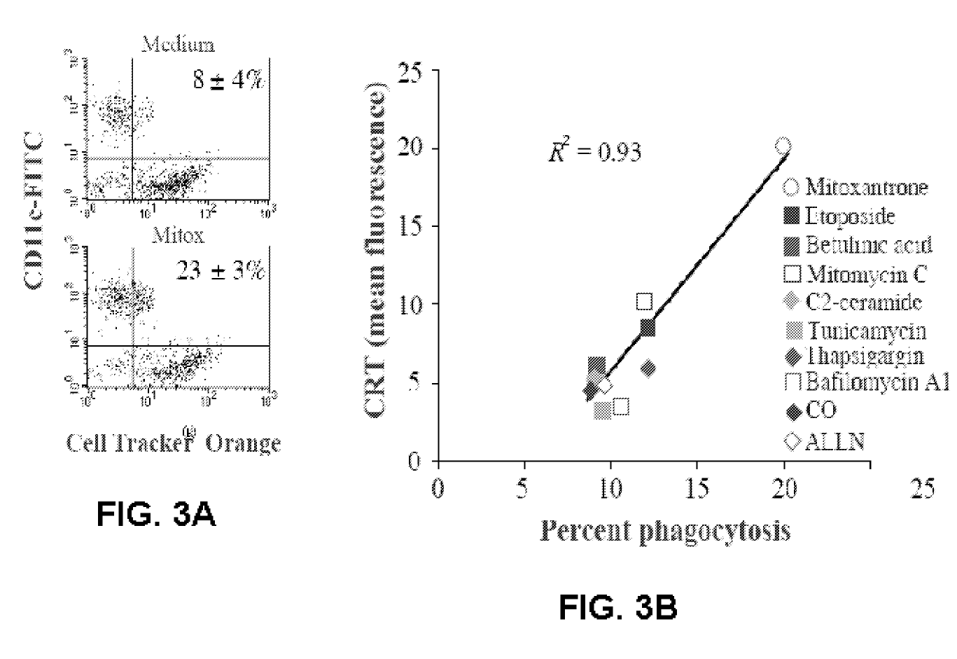

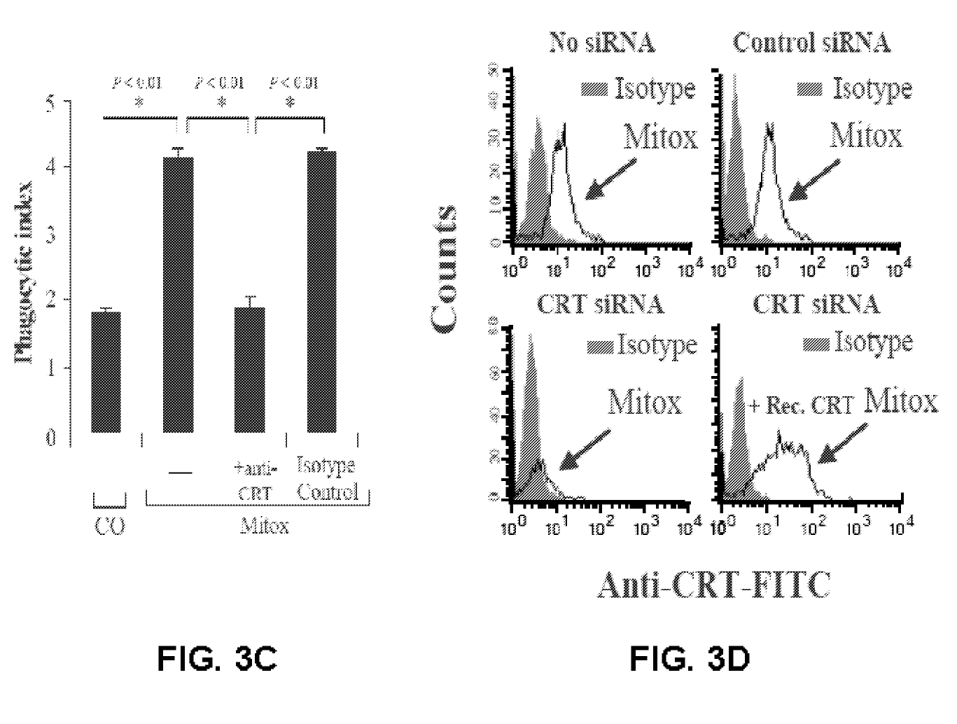

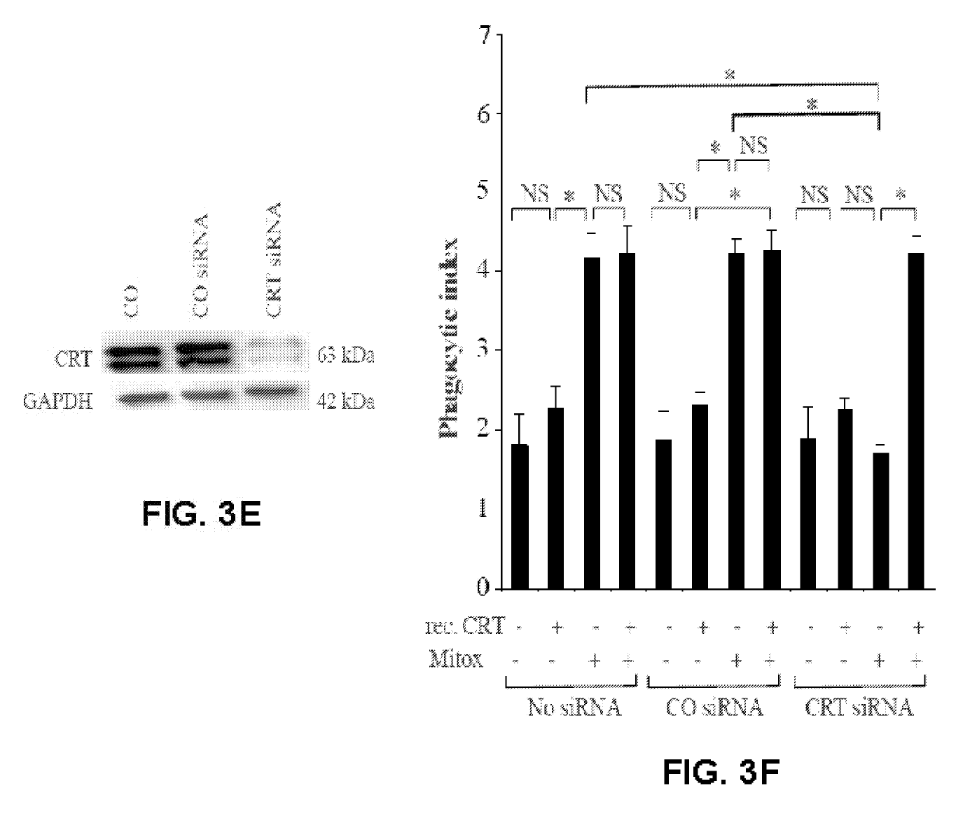

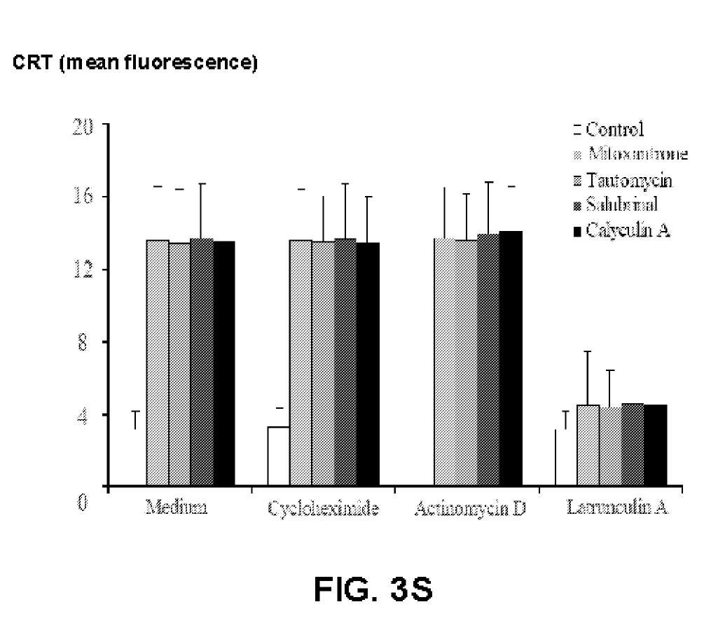

In view of the established role of CRT as an “eat me” signal it was decided to further investigate the possible implication of CRT in the phagocytosis of anthracyclin-treated tumor cells by DC, a cell type that b stringently required for mounting an immune response against apoptotic tumor cells. Anthracyclin-treated tumor cells acquired the property to be phagocytosed by DC quickly, well before the manifestation of apoptotic changes, within a few hours after treatment with doxorubicin or mitoxantrone (FIG. 3A, FIG. 2S A), correlating with the rapid induction of CRT (FIG. 3B, FIG. 1 S A, B) and the acquisition of immunogenicity (FIG. 2S B). The presence of CRT on the surface of tumor cells treated with a panel of distinct cell death inducers strongly correlated with their DC-mediated phagocytosis, suggesting that CRT is important in mediating the uptake of tumor cells by DC (FIG. 3B). Accordingly, blockade of the CRT present on the surface of mitoxantrone-treated cancer cells by means of a specific antibody from avian origin (which cannot interact with mouse Fc receptors) inhibited their phagocytosis by DC (FIG. 3C).

Similarly, knockdown of CRT with a specific siRNA (FIGS. 3D, E) suppressed the phagocytosis of anthracyclin-treated tumor cells (FIG. 2F). Addition of recombinant CRT protein (rCRT), which binds to the surface of the cells, could reverse the defect induced by the CRT-specific siRNA, both at the level of CRT expression (FIG. 3E) and phagocytosis by DC (FIG. 3F). Of note, rCRT alone could not promote DC maturation ex vivo over a large range of concentrations. Hence, surface CRT elicits phagocytosis by DC.

### Example 3

### Requirement of CRT for Immunogenicity of Dying Tumor Cells

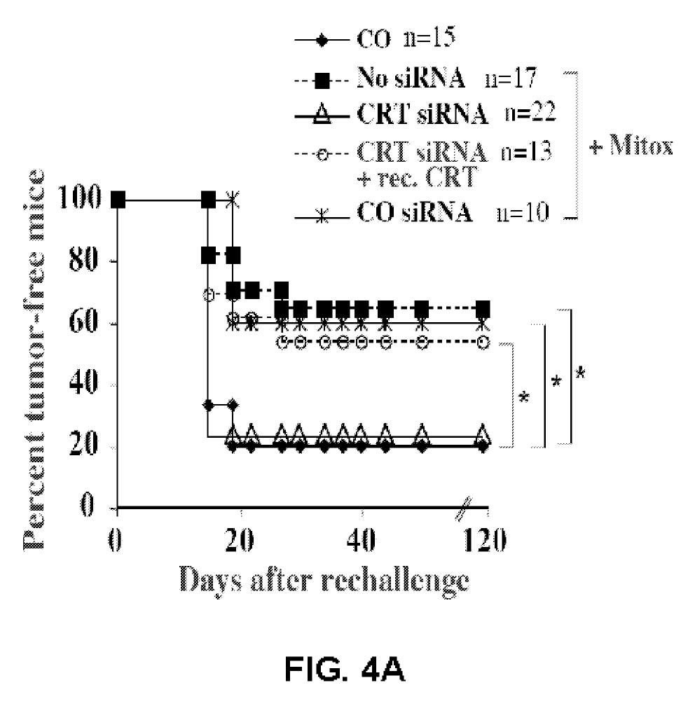

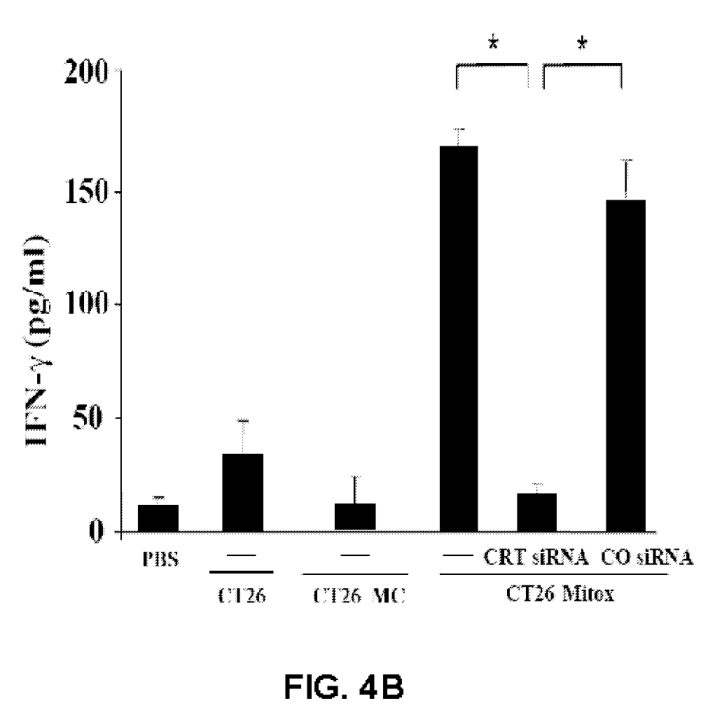

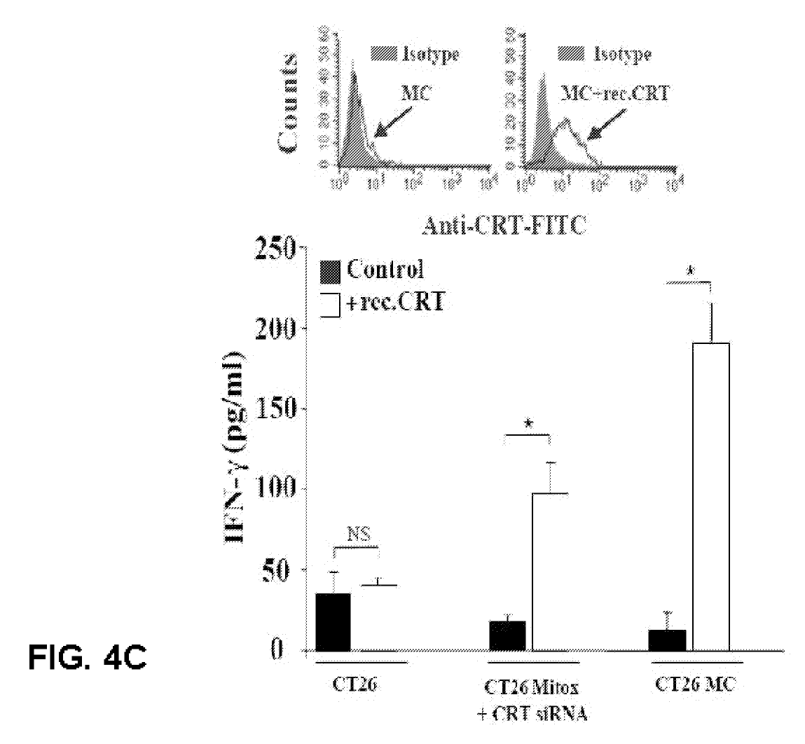

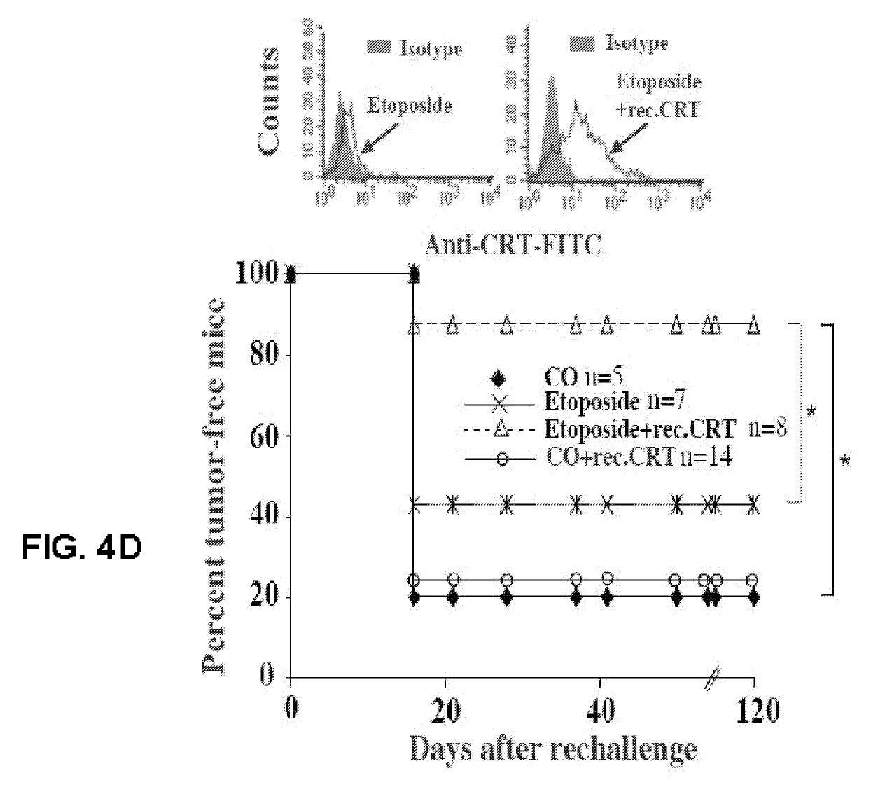

The knock-down of CRT compromised the immunogenicity of mitoxantrone-treated CT26 cells, and this defect was restored when rCRT was used to complement the CRT defect induced by the CRT-specific siRNA. This result was obtained in two distinct experimental systems, namely (i) when CT26 tumor cells were injected into the flank of Balb/c mice (or MCA205 cells were injected into C57BI/6 mice) to assess the efficacy of anti-tumor vaccination (FIG. 4A) and (ii) when the turn or cells were injected into the foot pad to measure interferon-7 production by T cells from the popliteal lymph node (FIG. 4B). In this latter system, absorption of rCRT to the plasma membrane surface greatly enhanced the immunogenicity of cells that usually fail to induce an immune response such as mitomycin-treated cells (FIG. 4C). Similarly, etoposide-treated cells coated with rCRT elicited a vigorous anti-tumor immune response in vivo, in conditions in which sham-coated cells treated with etoposide were poorly immunogenic (FIG. 4E). However, absorption of rCRT to the cell surface without prior treatment with cell death inducers failed to elicit an anti-cancer immune response and live rCRT-pretreated cells inoculated into mice formed tumors, both in immunocompetent (FIG. 4E) and immunodeficient mice (not shown). Thus, CRT critically determines the immunogenicity of cell death in vivo but does not determine cell death as such.

### Example 4

### Inhibitors of PP1/GADD34 Induce CRT Exposure and Induce Immunogenicity

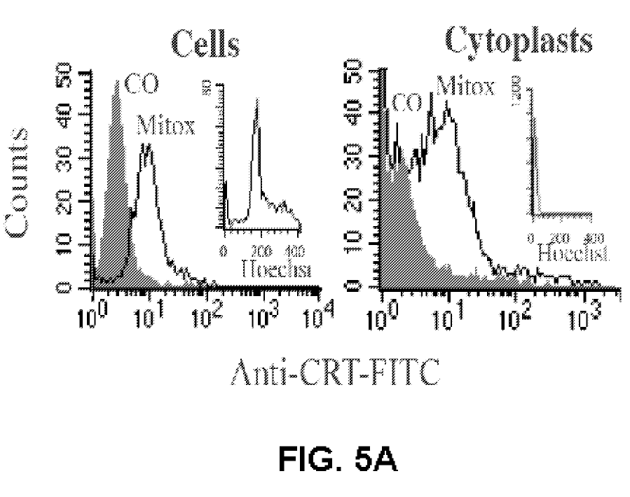

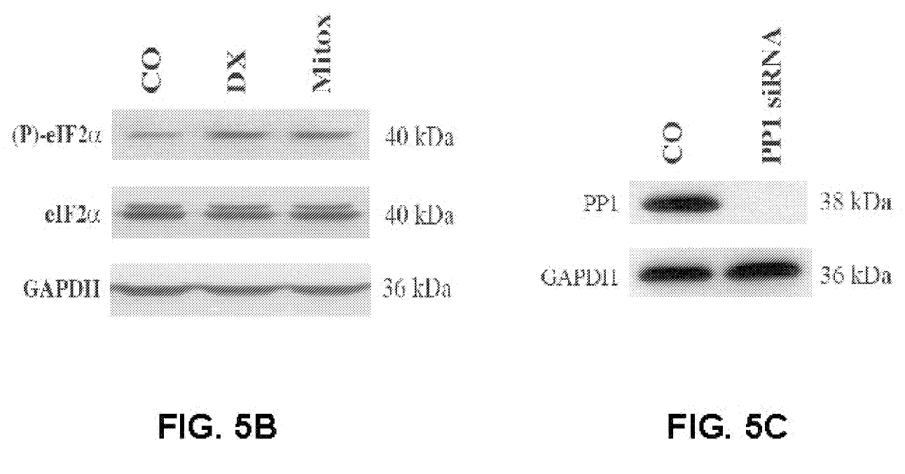

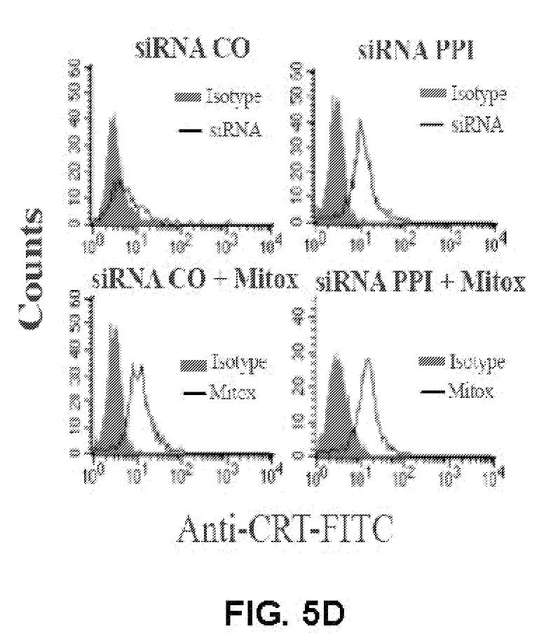

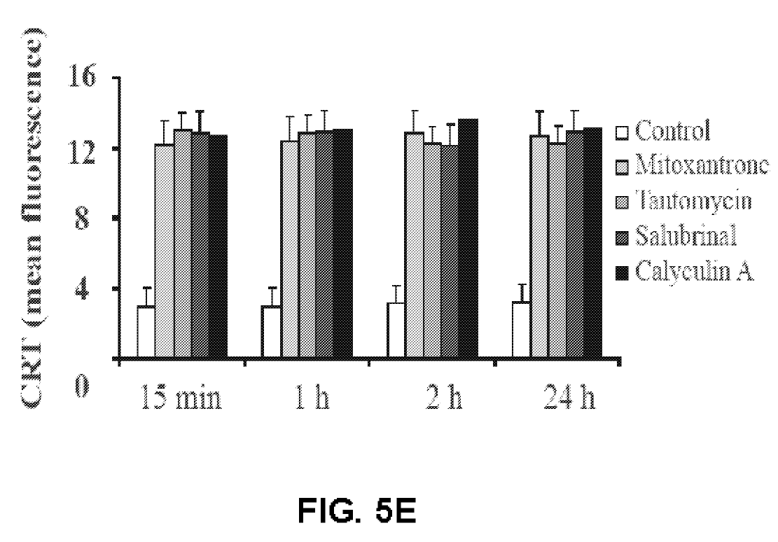

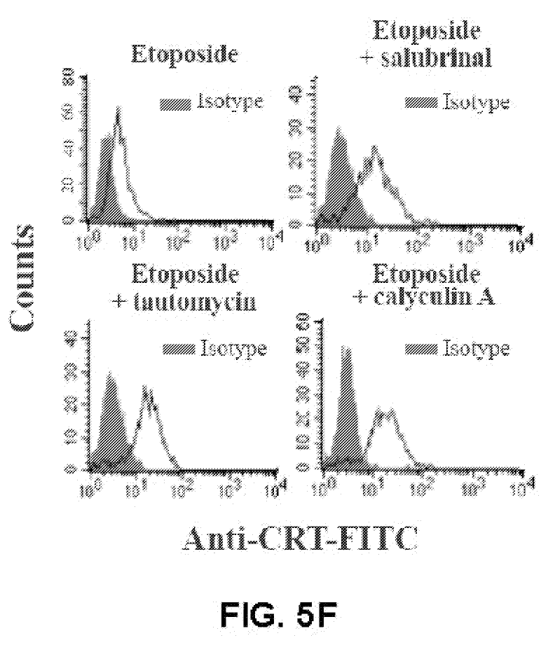

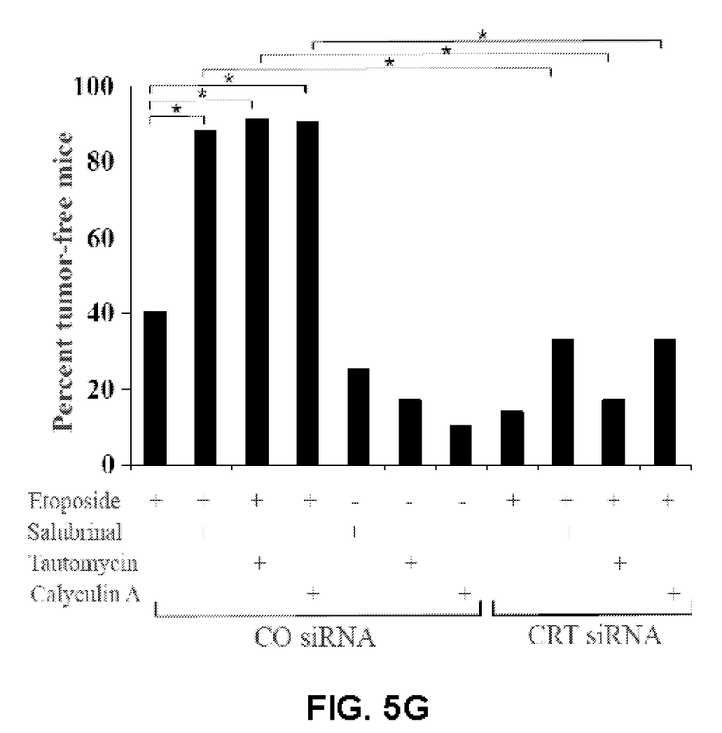

Since anthracyclin-induced CRT exposure was a rather rapid process (within 1 hour. FIGS. 1 SA, 1 SB), it was suspected that anthracyclins might exert effects that are not mediated by genotoxic stress. In response to mitoxantrone, enucleated cells (cytoplasts) readily (within 1 hour) exposed CRT (FIG. 5A) and became preys of DC as efficiently as intact cells (FIG. 3A), indicating the existence of a cytoplasmic (non-nuclear) anthracyclin target. Anthracyclins failed to induce immediate mitochondrial stress, yet caused the rapid phosphorylation of eIF2α, (FIG. 5B), a protein that is typically hyperphosphorylated in ER stress due to the activation of stress kinases. Knock-down of the four kinases known to phosphorylate eIF2α (GCN2, HRI, PERK, PKR) failed to inhibit the anthracyclin-stimulated CRT exposure. In contrast, knock-down of either GADD34 or the catalytic subunit of protein phosphatase 1 (PP1) (FIG. 5C), which together form the PP1/GADD34 complex involved in the dephosphorylation of eIF2α was sufficient to induce CRT exposure (FIG. 5D and not shown). The CRT exposure triggered by PP1 or GADD34 depletion was not further enhanced by mitoxantrone (FIG. 5D), suggesting that PP1/GADD34 and anthracyclins act on the same pathway to elicit CRT translocation to the cell surface. CRT exposure was efficiently induced by chemical PP1/GADD34 inhibitors, namely tautomycin, calyculin A (which both inhibit the catalytic subunit of PP1), as well as by salubrinal (which inhibits the PP1/GADD34 complex) (FIG. 5E). All these PP1/GADD34 inhibitors induced CRT exposure with a similar rapid kinetics as did anthracyclins, both in cells (FIG. 5E) and in cytoplasts.

Mitoxantrone and salubrinal induced CRT exposure on a panel of turn or cell lines from murine (MCA205, B16F10, J558) or human origin (HeLa, A549, HCT116). CRT exposure induced by anthracyclins and PP1/GADD34 inhibitors was not affected by inhibitors of transcription, translation or microtubuli, yet was abolished by latrunculin A, an inhibitor of the actin cytoskeleton and exocytosis (FIG. 33).

Inhibition of the PP1/GADD34 complex with salubrinal, calyculin A or tautomycin was not sufficient to induce immunogenic cell death (FIGS. 5F, G) (and the cells, which did not die, formed lethal tumors when injected into animals). However, these inhibitors greatly enhanced CRT exposure (FIG. 5F) and the immunogenic potential of cells succumbing to etoposide (FIG. 5G) or mitomycin C. This immunostimulatory effect was abrogated by knocking down CRT (FIG. 5G). Altogether, these results demonstrate that PP1/GADD34 inhibition induces CRT exposure, which, in turn, can stimulate the anti-tumor immune response.

### Example 5

### Immunogenic Chemotherapy by In Vivo Application of CRT or PP1/GADD34 Inhibitors

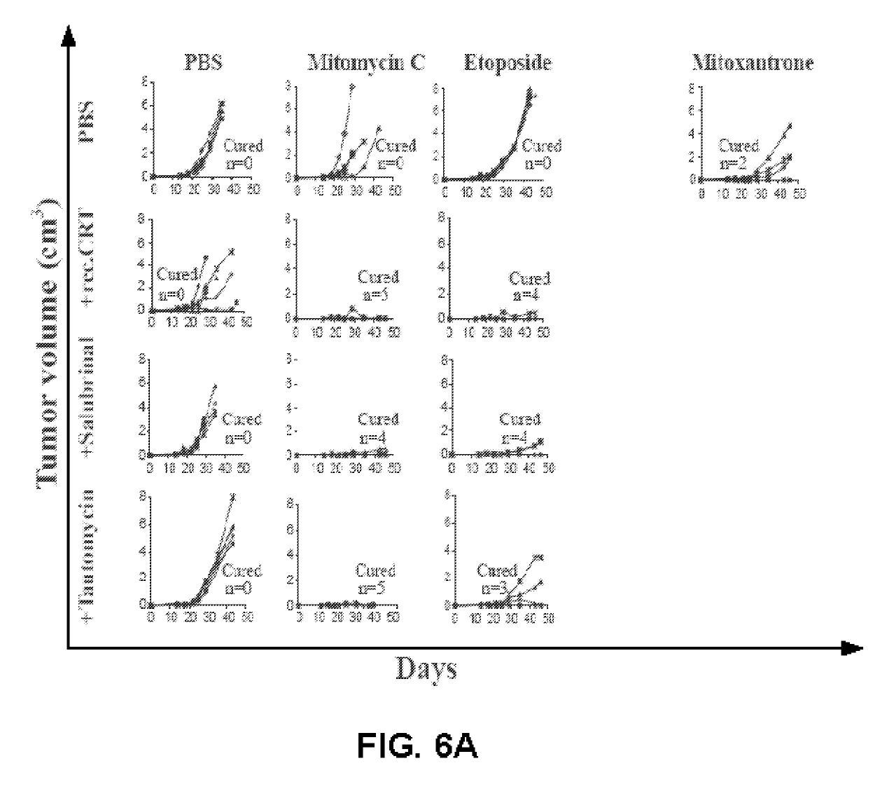

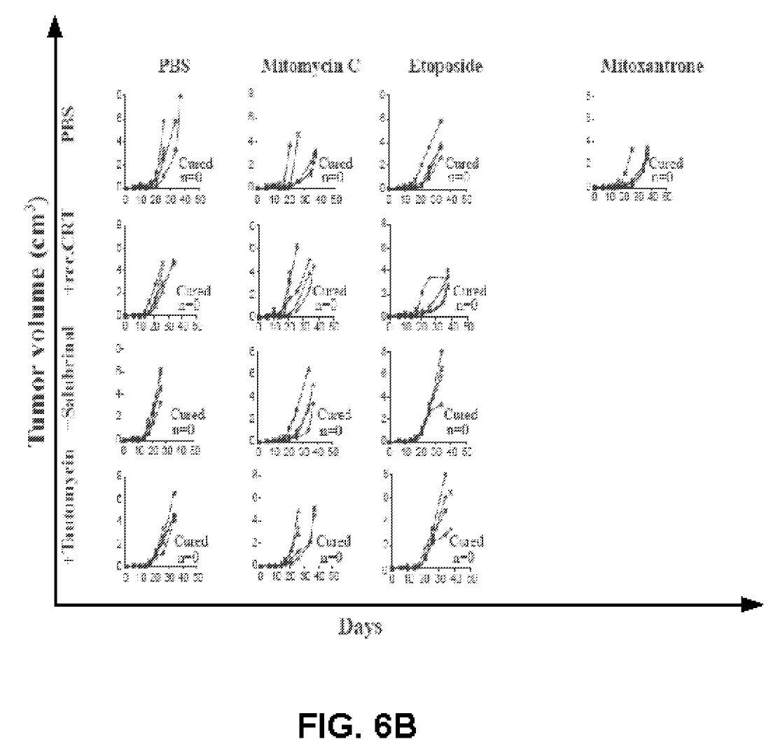

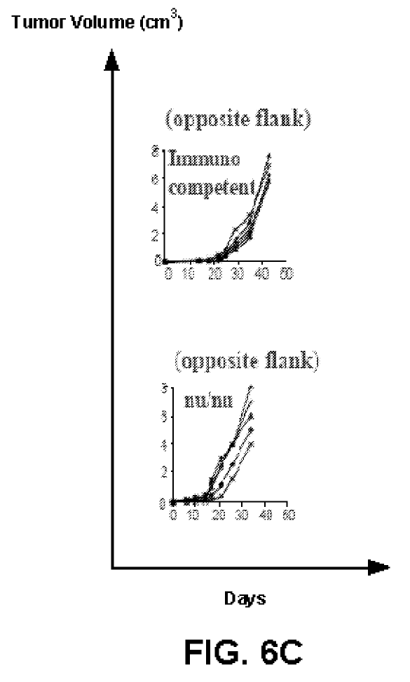

A single intratumoral injection of mitoxantrone into established 14-day-old CT26 tumors was able to cause their permanent regression in some but not all cases, if the tumors were established in immunocompetent BALB/c mice (FIG. 6A). However, there was no cure by mitoxantrone if the tumors were carried by immunodeficient nu/nu mice (FIG. 6B). The intratumoral injection of rCRT, salubrinal tautomycin, etoposide or mitomycin C had no major therapeutic effect, neither in immunocompetent nor in nu/nu mice. However, the combination of a cell death inducer (etoposide or mitomycin C) plus rCRT was able to cause tumor regression, in immunocompetent (but not in immunodeficient) animals. To obtain a therapeutic effect, rCRT had to be injected into the tumor. rCRT injected into a distant site did not ameliorate the antitumoral effects of intratumorally injected etoposide (FIG. 6C). Similarly, etoposide or mitomycin C could be combined with drugs that induce CRT exposure (salubrinal or tautomycin), leading to stable disease or complete tumor regression in immunocompetent (but not in athymic) hosts (FIG. 6A-B). Live CT26 cells failed to grow in animals that had been cured from CT26 tumors, indicating the establishment of a permanent anti-tumor immune response. Similar results were obtained when established MCA205 sarcomas (in C57BI/6 mice) or PRO colon carcinomas (in BDIX rats) were treated by local injections of weakly immunogenic cell death inducers plus rCRT or PP1/GADD34 inhibitors (not shown). These results delineate a strategy of immunogenic chemotherapy for the cure of established cancer.

1. A method of treating a disease in a mammal, comprising any one of: inducing a translocation of a protein to cellular membrane, or administering the protein from an extracellular medium to the cellular membrane, in order to provoke an immunogenic apoptosis.

2. The method of claim 1, wherein the protein includes any one or more of: endogenous calreticulin, recombinant calreticulin, and calreticulin in mimetic form.

3. The method of claim 2, wherein the disease includes any one or more of: cancer, autoimmune disorder, allergy, transplant rejection, and an infection.

4. The method of claim 3, wherein cancer includes any one of: breast cancer, prostate cancer, melanoma, colon cancer, lung cancer, kidney cancer, osteosarcoma, and a tumor sensitive to VP16/etoposide, radiotherapy, or immunotherapy.

5. The method of claim 3, wherein the infection includes any one of: a viral infection, a bacterial infection, a fungal infection, and a parasitic infection.

6. The method of claim 2, wherein treating the disease further includes using chemotherapy.

7. The method of claim 2, wherein inducing the translocation of calreticulin to the cellular surface comprises using of anthracyclin.

8. The method of claim 2, wherein the disease includes

9. The method of claim 1, wherein the mammal includes any one or more of: a mouse, a rat, and a human being.

10. The method of claim 5, wherein the anthracyclin b selected from any one or a combination of: doxorubicin, idarubicin, and mitoxantrone.

11. The method of claim 1, further comprising administering a cell-death inducer prior to inducing the translocation of the protein to the cellular membrane or administering the protein from the extracellular medium to the cellular membrane.

12. The method of claim 11, wherein the cell-death inducer includes any one of: etoposide and mitomycine C.

13. The method of claim 1, wherein the protein includes any one or more of: protein phosphatase inhibitor.

14. The method of claim 13, wherein the protein phosphatase inhibitor acts as a catalytic subunit of any one of: a protein phosphatase 1 (PP1) inhibitor, a GADD34 inhibitor, and a complex PP1/GADD34 inhibitor.

15. The method of claim 13, wherein the protein phosphatase inhibitor includes any one of: tautomycin, calyculin A, or salubrinal.

16. A kit for use in treating a disease in a mammal, by inducing a translocation of a protein to cellular membrane, or by administering the protein from an extracellular medium to the cellular membrane, in order to provoke an immunogenic apoptosis, comprising: detecting the level of protein presence at the cellular membrane, by detecting antibodies

17. The kit of claim 16, wherein the detected antibodies include anti-calreticulin antibodies that assist in detecting calreticulin protein at the cellular membrane.

18. The kit of claim 16, wherein the detected antibodies include anti-calreticulin antibodies that assist in predicting any one or more of an immunogenic viral infection, an autoimmune disease, a transplantation rejection, and a GVH disease.

19. A pharmaceutical composition for use in treating a disease in a mammal, by inducing a translocation of a protein to cellular membrane, or by administering the protein from an extracellular medium to the cellular membrane, in order to provoke an immunogenic apoptosis, comprising: a chemotherapeutic agent and recombinant calreticulin as a combination product.

20. The pharmaceutical composition of claim 19, wherein the combination product comprises a kinase activator.


SEQUENCE LISTING (2 sequences)

SEQ ID NO: 1 - protein, 416 aa, mus musculus
MLLSVPLLLGLLGLAAADPAIYFKEQFLDGDAWTNRWVESKHKSDFGKFVLSSGKFYGDLEKDKGLQTSQDARFYALSAKFEPFSNKGQTLVVQFTVKHEQNIDCGGGYVKLFPSGLDQKDMHGDSEYNIMFGPDICGPGTKKVHVIFNYKGKNVLINKDIRCKDDEFTHLYTLIVRPDNTYEVKIDNSQVESGSLEDDWDFLPPKKIKDPDAAKPEDWDERAKIDDPTDSKPEDWDKPEHIPDPDAKKPEDWDEEMDGEWEPPVIQNPEYKGEWKPRQIDNPDYKGTWIHPEIDNPEYSPDANIYAYDSFAVLGLDLWQVKSGTIFDNFLITNDEAYAEEFGNETWGVTKAAEKQMKDKQDEEQRLKEEEEDKKRKEEEEAEDKEDDDDRDEDEDEEDEKEEDEEESPGQAKDEL

SEQ ID NO: 2 - protein, 505 aa, mus musculus
MRFSCLALLPGVALLLASARLAAASDVLELTDENFESRVSDTGSAGLMLVEFFAPWCGHCKRLAPEYEAAATRLKGIVPLAKVDCTANTNTCNKYGVSGYPTLKIFRDGEEAGAYDGPRTADGIVSHLKKQAGPASVPLRTEEEFKKFISDKDASVVGFFRDLFSDGHSEFLKAASNLRDNYRFAHTNIESLVKEYDDNGEGITIFRPLHLANKFEDKTVAYTEKKMTSGKIKKFIQDSIFGLCPHMTEDNKDLIQGKDLLTAYYDVDYEKNAKGSNYWRNRVMMVAKKFLDAGHKLNFAVASRKTFSHELSDFGLESTTGEVPVVAIRTAKGEKFVMQEEFSRDGKALEQFLQEYFDGNLKRYLKSEPIPESNEGPVKVVVAENFDDIVNEEDKDVLIEFYAPWCGHCKNLEPKYKELGEKLSKDPNIVIAKMDATANDVPSPYEVKGFPTIYFSPANKKLTPKKYEGGRELNDFISYLQREATNPPIIQEEKPKKKKKAQEDL